SEQUENCE LISTING (26 sequences)

SEQ ID NO: 1 - RNA, 101 nt, Rattus norvegicus
  misc_feature (59)...(80): miR-132 mature sequence
ccgcccccgcgucuccagggcaaccguggcuuucgauuguuacugugggaaccggagguaacagucuacagccauggucgccccgcagcacgcccacgcuc

SEQ ID NO: 2 - RNA, 111 nt, Rattus norvegicus
  misc_feature (72)...(93): miR-212 mature sequence
cgggauauccccgcccgggcagcgcgccggcaccuuggcucuagacugcuuacugcccgggccgcccucaguaacagucuccagucacggccaccgacgccuggccccgcc

SEQ ID NO: 3 - RNA, 101 nt, Homo sapiens
  misc_feature (59)...(80): miR-132 mature sequence
ccgcccccgcgucuccagggcaaccguggcuuucgauuguuacugugggaacuggagguaacagucuacagccauggucgccccgcagcacgcccacgcgc

SEQ ID NO: 4 - RNA, 110 nt, Homo sapiens
  misc_feature (71)...(91): miR-212 mature sequence
cggggcaccccgcccggacagcgcgccggcaccuuggcucuagacugcuuacugcccgggccgcccucaguaacagucuccagucacggccaccgacgccuggccccgcc

SEQ ID NO: 5 - RNA, 66 nt, Mus musculus
  misc_feature (42)...(63): miR-132 mature sequence
gggcaaccguggcuuucgauuguuacugugggaaccggagguaacagucuacagccauggucgccc

SEQ ID NO: 6 - RNA, 91 nt, Mus musculus
  misc_feature (56)...(77): miR-212 mature sequence
gggcagcgcgccggcaccuuggcucuagacugcuuacugcccgggccgccuucaguaacagucuccagucacggccaccgacgccuggccc

SEQ ID NO: 7 - RNA, 101 nt, Macaca mulatta; identical to SEQ ID NO: 3
  misc_feature (9)...(80): miR-132 mature sequence

SEQ ID NO: 8 - RNA, 110 nt, Macaca mulatta
  misc_feature (71)...(91): miR-212 mature sequence
cggggcaccccgcccggacagcgcgccggcaccuuggcucuagacugcuuacugcccgggccgcccucaguaacagucuccagucagggccaccgacgccuggccccgcc

SEQ ID NO: 9 - RNA, 60 nt, Canis familiaris
  misc_feature (38)...(60): miR-132 mature sequence
aaccguggcuuucgauuguuacugugggaaccggagguaacagucuacagccauggucgc

SEQ ID NO: 10 - RNA, 62 nt, Canis familiaris
  misc_feature (1)...(23): miR-212 mature sequence
accuuggcucuagacugcuuacugcccgggccgcccucaguaacagucuccagucacggcca

SEQ ID NO: 11 - RNA, 66 nt, Monodelphis domestica
  misc_feature (42)...(63): miR-132 mature sequence
gggcaaccguggcuuucgauuguuacugugggaaccagggguaacagucuacagccauggucgccc

SEQ ID NO: 12 - RNA, 91 nt, Monodelphis domestica
  misc_feature (56)...(76): miR-212 mature sequence
gggcagcgcgccggcaccuuggcucuagacugcuuacugcccgggccacccucaguaacagucuccagucacggccaccgacgccuggccc

SEQ ID NO: 13 - RNA, 22 nt, Unknown
  Mature miR-132
uaacagucuacagccauggucg

SEQ ID NO: 14 - RNA, 21 nt, Unknown
  Mature miR-212
uaacagucuccagucacggcc

SEQ ID NO: 15 - RNA, 24 nt, Unknown
  Mature miR-217
uacugcaucaggaacugauuggau

SEQ ID NO: 16 - RNA, 22 nt, Unknown
  Mature miR-375
uuuguucguucggcucgcguga

SEQ ID NO: 17 - DNA, 50 bp, Artificial Sequence
  miR-132/RT linker probe
gtcgtatccagtgcagggtccgaggtattcgcactggatacgaccgacca

SEQ ID NO: 18 - DNA, 22 bp, Artificial Sequence
  miR-132/forward primer
gccgctaacagtctacagccat

SEQ ID NO: 19 - DNA, 16 bp, Artificial Sequence
  miR-132/reverse primer (universal primer)
gtgcagggtccgaggt

SEQ ID NO: 20 - DNA, 17 bp, Artificial Sequence
  miR-132/detector probe
tggatacgaccgaccat

SEQ ID NO: 21 - DNA, 50 bp, Artificial Sequence
  miR-212/RT linker probe
gtcgtatccagtgcagggtccgaggtaggcgcactggatacgacggccgt

SEQ ID NO: 22 - DNA, 21 bp, Artificial Sequence
  miR-212/forward primer
gccgctaacagtctccagtca

SEQ ID NO: 23 - DNA, 17 bp, Artificial Sequence
  miR-212/detector probe
tggatacgacggccgtg

SEQ ID NO: 24 - DNA, 55 bp, Artificial Sequence
  miR-375/RT linker probe
gtcgtatccagtgcagggtccgaggtaggcgcactggatacgacggccgttcacg

SEQ ID NO: 25 - DNA, 19 bp, Artificial Sequence
  miR-375/forward primer
ggcctttgttcgttcggct

SEQ ID NO: 26 - DNA, 17 bp, Artificial Sequence
  miR-375/detector probe
tggatacgactcacgca

(19) United States
(12) Patent Application Publication
(10) Pub. No.: US 2012/0034608 A1
(43) Pub. Date: Feb. 9, 2012

(54) MICRORNA AS A BIOMARKER OF PANCREATIC ISLET BETA-CELL ENGAGEMENT

(75) Inventors: Yun-Ping Zhou, East Brunswick, NJ (US); Andrew D. Howard, Park Ridge, NJ (US); Jin Shang, Short Hills, NJ (US)

(73) Assignee: Merck Sharp & Dohme Corp., Rahway, NJ (US)

(21) Appl. No.: 13/263,427

(22) PCT Filed: Apr. 5, 2010
(86) PCT No.: PCT/US10/29887

§ 371 Date: Oct. 7, 2011

Related U.S. Application Data
(60) Provisional application No. 61/212,636, filed on Apr. 14, 2009.

(51) Int. Cl. C12Q 1/68

(52) U.S. Cl. 435/6.11; 435/6.12; 435/6.13

(57) ABSTRACT

MicroRNAs (miRNAs) are short non-coding RNAs that regulate gene expression and which play important roles in many cell types, including as described herein, the pancreatic β-cell. Glucagon like peptide-1 (GLP-1), a hormone released from intestinal L-cells following meal intake, exerts pleiotropic effects on β-cell function including raising intracellular cAMP levels and now represents an important therapy for type 2 diabetes. Expression of miR-132 and miR212 is upregulated by CREB protein in response increased cAMP levels in the cell; therefore, methods for detecting and evaluating β-cell engagement by GLP-1 receptor agonists by monitoring miR-132 and miR-212 expression in a subject is described. The methods herein are particularly useful in the context of longitudinal clinical trials, such as those designed for testing the durability of any single or combination therapy in type 2 diabetes populations. Because the expression of these miRNAs is not affected by glucose, fatty acid, insulin, or β-cell function, monitoring miR-132 and miR-212 expression can be used to monitor the efficacy of any agent that effects an increase cAMP in β-cells. Such agents include for example, GLP-1, glucagon, GPR-119, and GIP receptor agonists; dipeptidyl peptidase IV (DPP IV) inhibitors; and phosphodiesterase inhibitors.

### BACKGROUND OF THE INVENTION

(1) Field of the Invention

The present invention relates to methods for detecting and evaluating pancreatic islet β-cell engagement by GLP-1, glucagon, GPR-119, and/or GIP receptor agonists administered to a subject by monitoring miR-132 and miR-212 expression in a test sample obtained from the subject. The methods herein are particularly useful in the context of longitudinal clinical trials, such as those designed for testing the durability of any single or combination therapy in type 2 diabetes populations. The expression of miR-132 and miR-212 is induced by cAMP-response element binding (CREB) protein in response to elevated cAMP and is not affected by glucose, fatty acid, insulin, or β-cell function. Therefore, monitoring miR-132 and miR-212 expression can be used to monitor the efficacy of any agent for treating a metabolic disorder that effects an increase in cAMP levels in β-cells. In addition to the above G protein-coupled receptors, such agents further include dipeptidyl peptidase IV (DPP IV) inhibitors and phosphodiesterase inhibitors.

(2) Description of Related Art

Diabetes mellitus can be divided into two clinical syndromes, Type I and Type II diabetes mellitus. Type I diabetes, or insulin-dependent diabetes mellitus, is a chronic autoimmune disease characterized by the extensive loss of β-cells in the pancreatic islets of Langerhans (hereinafter referred to as “pancreatic islet cells” or “islet cells”), which produce insulin. As these cells are progressively destroyed, the amount of secreted insulin decreases, eventually leading to hyperglycemia (abnormally high level of glucose in the blood) when the amount secreted drops below the level required for euglycemia (normal blood glucose level). Although the exact trigger for this immune response is not known, patients with Type I diabetes have high levels of antibodies against pancreatic β-cells (hereinafter “β-cells”). However, not all patients with high levels of these antibodies develop Type I diabetes.

Type II diabetes, or non-insulin-dependent diabetes mellitus, develops when muscle, fat, and liver cells fail to respond normally to insulin. This failure to respond (called insulin resistance) may be due to reduced numbers of insulin receptors on these cells or a dysfunction of signaling pathways within the cells, or both. The β-cells initially compensate for this insulin resistance by increasing their insulin output. Over time, these cells become unable to produce enough insulin to maintain normal glucose levels, indicating progression to Type II diabetes (Kahn Am. J. Med. 108 Suppl. 6a: 2S-8S (2000).

The fasting hyperglycemia that characterizes Type II diabetes occurs as a consequence of the combined lesions of insulin resistance and β-cell dysfunction. The β-cell defect has two components: the first component, an elevation of basal insulin release (occurring in the presence of low, non-stimulatory glucose concentrations), is observed in obese, insulin-resistant pre-diabetic stages as well as in Type II diabetes. The second component is a failure to increase insulin release above the already elevated basal output in response to a hyperglycemic challenge. This lesion is absent in pre-diabetes and appears to define the transition from normo-glycemic insulin-resistant states to diabetes. There is currently no cure for diabetes.

Conventional treatments for diabetes are very limited and focus on attempting to control blood glucose levels in order to minimize or delay complications. Current treatments target either insulin resistance (metformin, thiazolidinediones (TZDs)) or insulin release from the β-cell (sulphonylureas, exanatide). Sulphonylureas, and other compounds that act by depolarizing the β-cell have the side effect of hypoglycemia since they cause insulin secretion independent of circulating glucose levels. One approved drug, BYETTA (exanatide) stimulates insulin secretion only in the presence of high glucose. JANUVIA (sitagliptin) is another recently approved gliptin drug that increases blood levels of incretin hormones, which in turn can increase insulin secretion, reduce glucagon secretion, and effect an increase in intracellular levels of cAMP in β-cells.

Progressive insulin resistance and loss of insulin secreting pancreatic β-cells are primary characteristics of Type II diabetes. Normally, a decline in the insulin sensitivity of muscle and fat is compensated for by increases in insulin secretion from the β-cell. However, loss of β-cell function and mass results in insulin insufficiency and diabetes (Kahn, Cell 92: 593-596 (1998); Cavaghan et al., J. Clin. Invest. 106: 329-333 (2000); Saltiel, Cell 104: 517-529 (2001); Prentki & Nolan, J. Clin. Invest. 116: 1802-1812. (2006); and Kahn, J. Clin. Endocrinol. Metab. 86: 4047-4058 (2001)). Hyperglycemia further accelerates the decline in β-cell function (UKPDS Group, J.A.M.A. 281: 2005-2012 (1999); Levy et al., Diabetes Med. 15: 290-296, (1998); and Zhou et al., J. Biol. Chem. 278: 51316-23 (2003)).

Insulin secretion from the β-cells of pancreatic islets is elicited by increased levels of blood glucose. Glucose is taken up into the β-cell primarily by the β-cell and liver selective transporter GLUT2 (Thorens, Mol. Membr. Biol. 18(4): 265-73 (2001)). Once inside the cell, glucose is phosphorylated by glucokinase, which is the primary glucose sensor in the β-cell since it catalyzes the irreversible rate limiting step for glucose metabolism (Matschinsky, Curr. Diab. Rep. 5(3): 171-6 (2005)). The rate of glucose-6-phosphate production by glucokinase is dependent on the concentration of glucose around the β-cell and; therefore, this enzyme allows for a direct relationship between level of glucose in the blood and the overall rate of glucose oxidation by the cell. Mutations in glucokinase produce abnormalities in glucose dependent insulin secretion in humans giving further evidence that this hexokinase family member plays a key role in the islet response to glucose (Gloyn et al., J. Biol. Chem. 280(14): 14105-13 (2005)). Small molecule activators of glucokinase enhance insulin secretion and may provide a route for therapeutic exploitation of the role of this enzyme (Guertin & Grimsby, J. Curr Med. Chem. 13(15): 1839-43 (2006); and Matschinsky et al., Diabetes 55(1):1-12 (2006)) in diabetes. Glucose metabolism via glycolysis and mitochondrial oxidative phosphorylation ultimately results in ATP production and the amount of ATP produced in a β-cell is directly related to the concentration of glucose to which the β-cell is exposed.

Incretin hormones such as Glucagon-Like Peptide 1 (GLP-1) and Glucose-dependent Insulinotropic Polypeptide (GIP, also known as Gastric Inhibitory Polypeptide) also bind to specific Gα-coupled GPCR receptors on the surface of islet cells, including β-cells, and raise intracellular cAMP (Drucker, 3. Clin. Invest. 117(1): 24-32 (2007)). Although the receptors for these hormones are present in other cells and tissues, the overall sum of effects of these peptides appear to be beneficial to control of glucose metabolism in the organism (Hansotia et al., J. Clin. Invest. 117(1):143-52 (2007). GIP and GLP-1 are produced and secreted from intestinal K and L cells, respectively, and these peptide hormones are released in response to meals by both direct action of nutrients in the gut lumen and neural stimulation resulting from food ingestion. GIP and GLP-1 have short half-lives in human circulation due to the action of the protease dipeptidyl-peptidase IV (DPP IV) and inhibitors of this protease can lower blood glucose due to their ability to raise the levels of active forms of the incretin peptides. Peptides (e.g. exanatide (BYETTA)) and peptide-conjugates that bind to the GIP or GLP-1 receptors but are resistant to serum protease cleavage can also lower blood glucose substantially (Gonzalez et al., Expert Opin. Investig. Drugs 15(8): 887-95 (2006)). The clinical success of DPPIV inhibitors and incretin mimetics do point to the potential utility of compounds that increase incretin activity in the blood or directly stimulate cAMP production in the β-cell. Some studies have indicated that β-cell responsiveness to GIP is diminished in Type II diabetes (Nauck et al., J. Clin. Invest. 91: 301-307 (1993); and Elahi et al., Regul. Pept. 51: 63-74 (1994)). Restoration of this responsiveness (Meneilly et al., Diabetes Care 16(1): 110-4 (1993)) may be a promising way to improve β-cell function in vivo.

The GLP-1 receptor (GLP-1R) is a G protein-coupled receptor highly expressed in pancreatic β-cells. Upon activation, GLP-1R couples with G protein Gas, resulting in adenylate cyclase activation and cAMP elevation. This leads to cAMP dependent activation of PKA and Epac2, which regulates insulin secretion (Holz, Diabetes 53: 5-13 (2004): Kashima et al., J. Biol. Chem. 276: 46046-46053 (2001); Ozaki et al., Nat. Cell. Biol. 2: 805-811 (2000)). GLP-1R activation also induces IRS-2 and other gene expression pathways via ERK1/2, PKC and PI3K and promotes cell growth, differentiation and maintenance (Kim & Egan, ibid.; Park et al., J. Biol. Chem. 281: 1159-1168 (2006)). More recently, β-Arrestin-1 was shown to play a role in the GLP-1 signaling leading to enhanced insulin secretion (Sonoda et al., Proc. Natl. Acad. Sci. USA 105: 6614-9 (2008)). The molecular details downstream of these signaling pathways in 13-cell remain to be fully understood.

Incretins such as GLP-1 and GIP can also increase the rate of β-cell proliferation and decrease the apoptotic rates of β-cells in animal models (Farilla et al., Endocrinol. 143(11): 4397-408 (2002)) and human islets in vitro (Farilla et al., Endocrinol. 144(12): 5149-58 (2003)). The net result of these changes is an increase in β-cell number and islet mass, and this should provide for increased insulin secretory capacity, which is another desired aim of anti-diabetic therapies. GLP-1 has also been shown to protect islets from the destructive effects of agents such as streptozotocin by blocking apoptosis (Li et al., J. Biol. Chem. 278(1): 471-8 (2003)). Cyclin D1, a key regulator of progression through the cell cycle, is up-regulated by GLP-1 and other agents that increase cAMP and PKA activity also have a similar effect (Friedrichsen et al., J. Endocrinol. 188(3): 481-92 (2006); and Kim et al., J. Endocrinol. 188(3): 623-33 (2006)). Increased transcription of the cyclin D1 gene occurs in response to PKA phosphorylation of CREB (cAMP-response element binding) transcription factors (Hussain et al., Mol. Cell. Biol. 26(20): 7747-59 (2006)).

β-cell cAMP levels may also be raised by inhibiting cAMP to AMP degradation by phosphodiesterases (Furman & Pyne, Curr. Opin. Investig. Drugs 7(10):898-905 (2006)). There are several different cAMP phosphodiesterases in the β-cell and many of these have been shown to serve as a brake on glucose-dependent insulin secretion. While inhibitors of cAMP phosphodiesterases have been shown to increase insulin secretion in vitro and in vivo, including PDE1C, PDE3B, PDE10, (Han et al., J. Biol. Chem. 274(32): 22337-44 (1999); Harndahl et al., J. Biol. Chem. 277(40): 37446-55 (2002); Walz et al., J. Endocrinol. 189(3): 629-41 (2006); Choi et al., J. Clin. Invest. 116(12): 3240-51 (2006); and Cantin et al., Bioorg. Med. Chem. Lett. 17(10): 2869-73 (2007)), no PDEs have been found to have the cell type selectivity necessary to avoid undesirable effects. However, this remains an area of active investigation due to the potential for amplification of the effects of incretins and other agents that stimulate adenylate cyclase.

There appear to be multiple mechanisms by which cAMP elevation in the β-cell can enhance glucose dependent insulin secretion. Classically, many of the intracellular effects of cAMP are mediated by the cAMP-dependent protein kinase (protein kinase A, PKA) (Hatakeyama et al., J. Physiol. 570(Pt 2): 271-82 (2006)). PKA consists of a complex of two regulatory and two catalytic domains: binding of cAMP to the catalytic domains releases the catalytic domains and results in increased protein phosphorylation activity. One of the downstream effects of this kinase activity is enhanced efficiency of insulin exocytosis (Gromada et al., Diabetes 47(1): 57-65 (1998)). Another cAMP binding protein is Epac, a guanine nucleotide exchange factor (GEF) (Kashima et al., J. Biol. Chem. 276(49): 46046-53 (2001) and Shibasaki et al., J. Biol. Chem. 279(9): 7956-61 (2004)), which mediates a cAMP-dependent but PKA-independent increase in insulin exocytosis. Epac activated by cAMP may also enhance of release of intracellular Ca²⁺ (Holz, Diabetes 53(1): 5-13 (2004)). The effects of cAMP on insulin secretion are dependent on elevated glucose levels, so raising cAMP in the pancreatic β-cell is an important goal for therapeutics of Type II diabetes.

Agents that raise intracellular cAMP levels in the β-cell increase insulin secretion in a glucose dependent manner (Miura & Matsui, Am. J. Physiol. Endocrinol. Metab 285: E1001-E1009 (2003)). One mechanism for raising cAMP is by the action of G-protein coupled cell surface receptors, which stimulate the enzyme adenylate cyclase to produce more cAMP. The GLP-1 receptor is an example of such a receptor (Thorens et al., Diabetes 42: 1678-1682 (1993)). It is clear that many efficacious diabetic treatments will rely upon agents that raise intracellular concentrations of cAMP in β-cells. Thus, because there is a need for drugs for the treatment of diabetes that increase intracellular levels of cAMP in β-cells, there is also a need for a non-invasive method to determine whether such drugs are effectively engaging the β-cells.

### BRIEF SUMMARY OF THE INVENTION

MicroRNAs (miRNAs) are short non-coding RNAs that regulate gene expression and which play important roles in many cell types, including as described herein, the pancreatic β-cell. Glucagon like peptide-1 (GLP-1), a hormone released from intestinal L-cells following meal intake, exerts pleiotropic effects on β-cell function including raising intracellular cAMP levels and now represents an important therapy for type 2 diabetes. Expression of miR-132 and miR212 is upregulated by CREB protein in response increased cAMP levels in the cell. The present invention provides methods for detecting and evaluating β-cell engagement by GLP-1 receptor agonists by monitoring miR-132 and miR-212 expression in a subject. The methods herein are particularly useful in the context of longitudinal clinical trials, such as those designed for testing the durability of any single or combination therapy in type 2 diabetes populations. Because the expression of these miRNAs is not affected by glucose, fatty acid, insulin, or β-cell function, monitoring miR-132 and miR-212 expression can be used to monitor the efficacy of any agent that effects an increase cAMP in β-cells. Such agents include for example, GLP-1, glucagon, GPR-119, and GIP receptor agonists; dipeptidyl peptidase IV (DPP IV) inhibitors; and phosphodiesterase inhibitors.

Therefore, in one embodiment, the present invention provides a method for determining whether a treatment for a metabolic disorder that includes an agent that effects an increase in intracellular cAMP in pancreatic islet β-cells is engaging the pancreatic islet β-cells in a subject, comprising: measuring the level of at least one of an miRNA selected from the group consisting of miR-132 or miR-212 in a test sample from the subject undergoing the treatment, wherein an increase in the level of the miRNA in the test sample relative to the level of the corresponding miRNA in a control sample indicates the treatment is engaging the pancreatic islet β-cells.

In particular aspects, the miRNA is detected using reverse-transcription polymerase chain reaction (RT-PCR), particularly in assays in which the RT-PCR comprises obtaining total RNA from the test sample, adding the total RNA to a reaction mixture comprising (a) a linker probe having a stem, a loop, and a 3′ end sequence that base pairs with a 3′ end sequence of the miRNA, allowing the linker probe to hybridize the miR-132, and extending the linker probe to form an extension reaction product; (b) amplifying the extension product to produce an amplification product in a polymerase chain reaction comprising a forward primer that hybridizes to the 5′ region of the miRNA sequence in the extension or amplification product or a complementary sequence to the 5′ region of the miRNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and (c) detecting the detectable signal wherein the presence of the detectable signal relative to the level of the detectable signal in the control reaction indicates the agent is engaging the pancreatic islet β-cells.

In another embodiment, the present invention provides a method for determining whether a treatment for a metabolic disorder that includes an agent that effects an increase in intracellular cAMP in pancreatic islet β-cells is engaging the cells, comprising: (a) providing a test sample form a subject undergoing the treatment: (b) obtaining total RNA from the test sample and adding the total RNA to a reaction mixture comprising a linker probe having a stem, a loop, and a 3′ end sequence that base pairs with a 3′ end sequence of miR-132, allowing the linker probe to hybridize the miR-132, and extending the linker probe to form an extension reaction product; (c) amplifying the extension product to produce an amplification product in a polymerase chain reaction comprising a forward primer that hybridizes to the 5′ region of the miR-132 sequence in the extension or amplification product or a complementary sequence to the 5′ region of the miR-132 sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and (d) detecting the detectable signal wherein the presence of the detectable signal relative to the level of the detectable signal in a control reaction indicates the agent is engaging the pancreatic islet β-cells.

In further aspects of the above, the linker probe has the nucleotide sequence set forth in SEQ ID NO:17, the forward primer has the nucleotide sequence set forth in SEQ ID NO:18, the reverse primer has the nucleotide sequence set forth in SEQ ID NO:19, and the detector probe has the nucleotide sequence set forth in SEQ ID NO:20. In further still aspects, the method of claim 20, wherein the detector probe is conjugated to 6-carboxyfluorescein (6-FAM) at the 5′ end and an minor groove binder (MGB) ligand conjugated to tetramethylrhodamine (TAMRA) at the 3′ end.

In a further embodiment, the present invention provides a method for determining the efficacy of a treatment regimen for diabetes comprising: (a) obtaining a body fluid sample from a subject undergoing the treatment regimen; and (b) measuring the level of at least one of an miRNA selected from the group consisting of miR-132 or miR-212 in a test sample from the subject, wherein an increase in the level of the miRNA in the test sample relative to the level of the corresponding miRNA in a control sample indicates the treatment is efficacious.

In particular aspects, the miRNA is detected using reverse-transcription polymerase chain reaction (RT-PCR), particularly in assays in which the RT-PCR comprises obtaining total RNA from the test sample, adding the total RNA to a reaction mixture comprising (a) a linker probe having a stem, a loop, and a 3′ end sequence that base pairs with a 3′ end sequence of the miRNA, allowing the linker probe to hybridize the miR-132, and extending the linker probe to form an extension reaction product; (b) amplifying the extension product to produce an amplification product in a polymerase chain reaction comprising a forward primer that hybridizes to the 5′ region of the miRNA sequence in the extension or amplification product or a complementary sequence to the 5′ region of the miRNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and (c) detecting the detectable signal wherein the presence of the detectable signal relative to the level of the detectable signal in the control reaction indicates the treatment is efficacious.

In a further embodiment, the present invention provides a method for determining whether a treatment for a metabolic disorder that includes an agent that effects an increase in intracellular cAMP in pancreatic islet β-cells is engaging the cells, comprising: (a) providing a test sample form a subject undergoing the treatment: (b) obtaining total RNA from the test sample and adding the total RNA to a reaction mixture comprising a linker probe having a stem, a loop, and a 3′ end sequence that base pairs with a 3′ end sequence of miR-212, allowing the linker probe to hybridize the miR-212, and extending the linker probe to form an extension reaction product; (c) amplifying the extension product to produce an amplification product in a polymerase chain reaction comprising a forward primer that hybridizes to the 5′ region of the miR-212 sequence in the extension or amplification product or a complementary sequence to the 5′ region of the miR-212 sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and (d) detecting the detectable signal wherein the presence of the detectable signal relative to the level of the detectable signal in a control reaction indicates the agent is engaging the pancreatic islet β-cells.

In further aspects of the above, the linker probe has the nucleotide sequence set forth in SEQ ID NO:17, the forward primer has the nucleotide sequence set forth in SEQ ID NO:18, the reverse primer has the nucleotide sequence set forth in SEQ ID NO: 19, and the detector probe has the nucleotide sequence set forth in SEQ ID NO:20. In further still aspects, the method of claim 20, wherein the detector probe is conjugated to 6-carboxyfluorescein (6-FAM) at the 5′ end and an minor groove binder (MOB) ligand conjugated to tetramethylrhodamine (TAMRA) at the 3′ end.

In a further embodiment, the present invention provides a method for identifying an agent for treating a metabolic disorder that targets a receptor in pancreatic islet β-cells that raises intracellular levels of cAMP, comprising: measuring the level of at least one of an miRNA selected from the group consisting of miR-132 or miR-212 in a test sample obtained from a subject administered the agent, wherein an increase in the level of the miRNA in the test sample relative to the level of the corresponding miRNA in a control sample indicates the agent is targeting the receptor in the pancreatic islet β-cells that raises cAMP levels in the pancreatic islet β-cells.

In particular aspects, the miRNA is detected using reverse-transcription polymerase chain reaction (RT-PCR), particularly in assays in which the RT-PCR comprises obtaining total RNA from the test sample, adding the total RNA to a reaction mixture comprising (a) a linker probe having a stem, a loop, and a 3′ end sequence that base pairs with a 3′ end sequence of the miRNA, allowing the linker probe to hybridize the miR-132, and extending the linker probe to form an extension reaction product; (b) amplifying the extension product to produce an amplification product in a polymerase chain reaction comprising a forward primer that hybridizes to the 5′ region of the miRNA sequence in the extension or amplification product or a complementary sequence to the 5′ region of the miRNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and (c) detecting the detectable signal wherein the presence of the detectable signal relative to the level of the detectable signal in the control reaction indicates the agent is engaging the pancreatic islet β-cells.

In further aspects of the above, the linker probe has the nucleotide sequence set forth in SEQ ID NO:17, the forward primer has the nucleotide sequence set forth in SEQ ID NO:18, the reverse primer has the nucleotide sequence set forth in SEQ ID NO:19, and the detector probe has the nucleotide sequence set forth in SEQ ID NO:20. In further still aspects, the method of claim 20, wherein the detector probe is conjugated to 6-carboxyfluorescein (6-FAM) at the 5′ end and an minor groove binder (MGB) ligand conjugated to tetramethylrhodamine (TAMRA) at the 3′ end.

In further aspects of any one of the embodiments herein, the test sample is whole blood, plasma, or serum.

In particular aspects of any one of the embodiments herein, the agent is a glucagon-like peptide-1 (GLP-1) receptor agonist, a glucagon peptide receptor agonist, or is both a glucagon-like peptide-1 (GLP-1) receptor agonist and a glucagon peptide receptor agonist.

In particular aspects of any one of the embodiments herein, the agent is a glucagon-like peptide-1 (GLP-1) receptor agonist and a glucagon peptide receptor antagonist.

In particular aspects of any one of the embodiments herein, the agent is a glucose-dependent insulinotropic polypeptide (GIP) receptor agonist.

In particular aspects of any one of the embodiments herein, the agent is a G-protein-coupled receptor 119 (GPR-119) receptor agonist.

In particular aspects of any one of the embodiments herein, the agent is a phosphodiesterase inhibitor or dipeptidyl peptidase (DPP IV) inhibitor.

In further still aspects, the agent is selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, GPR-119 agonist, phosphodiesterase inhibitor, dipeptidyl peptidase (DPP IV) inhibitor, and combinations thereof.

In particular aspects of any one of the embodiments herein, the agent is an insulinotropic peptide selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, and combinations thereof.

The present invention is particularly useful for monitoring a metabolic disorder including but not limited to metabolic syndrome, obesity, diabetes (type I or type II), metabolic syndrome X, hyperglycemia, impaired fasting glucose, dyslipidemia, atherosclerosis, or other prediabetic state.

### BRIEF DESCRIPTION OF THE DRAWINGS

FIG. 1 shows miRNA expression profiling of GLP-1 treated INS-1 832/3 cells. miRNA profiling was performed by real-time qRT-PCR. Data represent the mean of three independent measurements with cells at different passages. The x-axis of the scatter plot represents the copy number of miRNA per 10 pg total RNA and the y-axis is the ratio of miRNA expression changes in response to 50 nM GLP-1 for 24 hours.

FIG. 2 shows a time course of microRNA induction by GLP-1 in INS-1 832/3 cells. miRNAs were measured by TAQMAN in INS-1 cells treated with GLP-1 (50 nM) for the periods of time as labeled. The expression levels were normalized to 4.5S RNA. Data represents mean±SE of three independent treatment groups.

FIG. 3 shows the induction of miR-132 and miR-212 expression by GLP-1 in rat islets. Islets were isolated from normal male Sprague-Dawley rats and cultured in RPMI 1640 medium with or without 50 nM GLP-1 for 24 hours. Levels of the MicroRNAs were determined by TAQMAN PCR and normalized to 4.5S RNA. Data are mean±SE of three independent islet preparations. *p<0.05.

FIG. 4 shows the release of miR-132 and miR-212 from the INS-1 832/3 cells cultured in the presence of 50 nM GLP-1. Levels of the microRNAs from culture media were determined by TAQMAN PCR and normalized to U6 RNA.

FIG. 5 shows that GLP-1 treatment increased plasma levels of miR-132 and miR-212 in C57/Bl6 mice.

FIG. 6 shows the effects of cAMP enhancing agents on miR-212 and miR-132 expression in INS-1 (832/3) cells. FIG. 6A: TAQMAN analysis of miR-132, miR-212, and miR-375 expression changes in response to 50 nM GLP-1, 50 nM Exendin-4, 5 μM IBMX (isobutylmethylxanthine) and 1 μM Foskolin (Fsk). FIG. 6B: cAMP accumulation over 1 hour after treatment of 50 nM GLP-1, 50 nM Exendin 4, or 1 μM Foskolin (Fsk). Data are the mean±SE of three independent treatment groups.

FIG. 7 shows overexpression of miR-132 and miR-212 enhanced GDIS in INS-1 832/3 cells. FIG. 7A measures insulin secretion: INS-1 832/3 cells were transfected with precursors for miR-132, miR-212, miR-375, or scramble control. Insulin secretion was measured 48 hours post-transfection in a 2 hour incubation in KRB media with 2 (G2), 8 (G8) or 16 (G16) mM glucose, or 30 mM KCl. FIG. 7C measures insulin content: insulin was measured from cell lysates and normalized to total protein. FIG. 7C shows the percentage of insulin release over insulin content. FIG. 7D shows TAQMAN quantification of insulin transcripts. Data are mean±SE of 5-6 replicates. *p<0.05, **p<0.01 as compared to the scramble control.

FIG. 8 shows the effects of over- and under-expression of miR-132 and miR-375 on GDIS and GLP-1 potentiation. FIG. 8A: Insulin secretion in INS-1 832/3 cells overexpressing miR-132 or miR-375. FIG. 8B: Insulin secretion in INS-1 cells under-expressing miR-132 or miR-375. 100 pmole pre-miRNA or anti-miRNA oligos were electroporated into the INS-1 cells and insulin secretion was measured 48 hour post transfection in a two-hour incubation in KRB medium with 2 (G2) or 16 mM glucose (G16), or G16+10 nM GLP-1. Data are mean±SE of 3 replicate. *p<0.05, **p<0.01 compared to scramble controls.

FIG. 9 shows INS-1 832/3 and 832/13 cells have distinct responsiveness to GLP-1. FIG. 9A shows GLP-1 potentiation of glucose-dependent insulin secretion by GLP-1. INS-1 832/13 and 832/3 cells were plated and cultured in 96-well plates to near confluency. Insulin secretion in response to GLP-1 was measured by a two-hour incubation in KRB medium with 16 mM glucose. Data are mean±SE of 3-5 replicates. FIG. 9B shows cAMP accumulation in 832/3 and 832/13 cells one hour post-treatment with Exendin-4 at 5.5 mM glucose. FIG. 9C shows up-regulation of miR-212 and miR-132 expression by GLP-1 is absent in INS-1 (832/13) cell. TAQMAN analysis was performed 24 hours post-GLP-1 treatment in triplicate. *p<0.05.

FIG. 10 shows the effects of over-expression and under-expression of miR-132 or miR-212 on insulin secretion in INS-1 832/13 cells. INS-1 832/13 cells were transfected with precursors or antisense inhibitors of miR-132 or pre-miR-212, or scramble control. Insulin secretion was measured under the same condition as described in FIG. 7. Data are mean±SE of 5-6 replicates. *p<0.05, **p<0.01 as compared to the scramble control.

### DETAILED DESCRIPTION OF THE INVENTION

The discovery of microRNAs (miRNAs) has revealed a new dimension of biological regulation downstream of signaling pathways (Bartel, Cell 116:281-297 (2004); Bartel, Cell 136: 215-233 (2009)). MicroRNAs typically comprise single-stranded, endogenous oligoribonucleotides of roughly 22 (18-25) nucleotides in length that are processed from larger stem-looped precursor RNAs. MicroRNAs appear to regulate gene expression by pairing to 3′ untranslated region sequences of target mRNAs and directing their post-transcriptional repression. Currently several hundreds of microRNAs have been identified in mammalian species with 286 in rats, 488 in mice and 695 in humans (Griffiths-Jones et al., Nucl. Acid Res. 36(Database Issue): D154-D158 (2008); Griffiths-Jones et al., Nucl. Acid Res. 34 (Database Issue): D140-D144 (2006); Griffiths-Jones, Nucl. Acid Res. 32(Database Issue): D109-D111 (2004); Ambros et al., RNA 9(3):277-279 (2003)). Previous studies by several groups have demonstrated that microRNAs, such as miR-375, may directly regulate both embryonic islet development and islet function in adult animals (Poy et al., Nature 432: 226-230 (2004); Kloosterman et al., PLoS Biol. 5: e203 (2007); Joglekar et al., Gene Expr. Patterns 9: 109-13 (2009).

A profile of miRNA expression in INS-1 832/3 cells (rat pancreatic beta-cell line that secretes insulin in response to glucose) identified two related microRNAs, miR-132 and miR-212, that were significantly up-regulated by GLP-1 stimulation (See Examples herein). This up-regulation was further confirmed in isolated rat islet cells. We show in the examples that over-expression of miR-132 or miR-212 significantly enhanced glucose-dependent insulin secretion and GLP-1 potentiation in the INS-1 832/3 cells. In contrast, the induction of miR-132 and miR-212 expression by GLP-1 was largely mitigated in INS-1 832/13 cells, a sibling line with poor insulin secretion and cAMP elevation in response to GLP-1. The results suggest that the insulinotropic effect of GLP-1 in pancreatic β-cell is mediated in part by cAMP-regulated induction of miR-132 and miR-212.

During recent years, a number of microRNAs have been implicated in β-cell function. Overexpression of miR-375 suppressed glucose-induced insulin secretion, and conversely, inhibition of endogenous miR-375 function in β-cells enhanced insulin secretion (Poy et al., Nature 432: 226-230 (2004)). In a more recent study, miR-375 was shown to directly target PDK1, resulting in decreased glucose-stimulatory action on insulin gene expression (El Ouaamari et al., Diabetes 57: 2708-17 (2008). In the Examples herein, miR-375 was used as a control for functional assays in INS-1 cells. The results in the Examples herein confirmed the above mentioned effects of miR-375 on glucose-dependent insulin secretion and insulin gene expression. In addition, unlike miR-132 and miR-212, miR-375 is not regulated by cAMP, thus it also served as a negative control for GLP-1 responsiveness in the experiments in the Examples.

In addition to miR-375, several other β-cell expressing microRNAs were reported to regulate the optimal production and secretion of insulin. MicroRNA miR-9 was reported to play a role in controlling insulin secretion via targeting Onecut-2 (Plaisance et al., J. Biol. Chem. 281: 26932-26942 (2006)). MicroRNA miR-124a was found to directly target FoxA2, which regulates the expression of several key β-cell genes including Pdx-1, Kir6.2, and Sur-1 (Baroukh et al., J. Biol. Chem. 282: 19575-19588 (2007)). Moreover, overexpression of miR-124a was shown to decrease GDIS by directly targeting Rab27A in MIN6B1 cells (Lovis et al., J. Biol. Chem. 389: 305-312, 2008)). More recently, miR-30d was reported to be a glucose-regulated microRNA that plays a role in regulating insulin transcription (Tang et al., RNA15: 287-293 (2009)). However, none of these microRNAs were found to be regulated by GLP-1. We show in the Examples that miR-132 and miR-212 are two new members of microRNAs that are involved in pancreatic β-cell function.

MicroRNA miR-132 expression, reported to be enriched in neurons and play a role in neuronal morphogenesis via targeting p250GAP, is regulated by cAMP-response element binding protein (CREB) (Vo et al., Proc. Natl. Acad. Sci. USA 102: 16426-31 (2005); Wayman et al., Proc. Natl. Acad. Sci. USA 105: 9093-8 (2008)). Moreover, methyl-CpG-binding protein 2 (MeCP2) is a target of miR-132 and both increase and decrease in MeCP2 cause neurodevelopmental defects (Klein et al., Nat. Neurosci. 10: 1513-151 (2007)). Comparative sequence analysis has revealed that miR-132 and miR-212 are closely related microRNA species: they are physically linked on chr17 in human genome, share identical seed region, and have a cAMP-response element (CRE) regulatory sequence in their promoter regions (Wu & Xie, Genome Biol. 7: R85 (2006)). Our results are the first to show the biological function of miR-132 and 212 in pancreatic β-cells, a non-neuronal cell type. As the examples herein suggest, insulin secretion enhanced by GLP-1 is mediated in part through induction of miR-132 and miR-212 expression in pancreatic β-cells, thus implicating these microRNAs as participants in the cAMP-dependent regulatory pathway by which the incretin GLP-1 exerts its effects in β-cells.

The nucleotide sequences for the rat miR-132 and miR-212 stem-loop precursor miRNAs are rno-mir-132 MI0000905: 5′-CCGCCCCCGC GUCUCCAGGG CAACCGUGGC UUUCGAUUGU UACUGUGGGA ACCGGAGGUA ACAGUCUACA GCCAUGGUCG CCCCGCAGCA CGCCCACGCU C-3′ (SEQ ID NO:1) and rno-mir-212 MI0000952: 5′-CGGGAUAUCC CCGCCCGGGC AGCGCGCCGG CACCUUGGCU CUAGACUGCU UACUGCCCGG GCCGCCCUCA GUAACAGUCU CCAGUCACGG CCACCGACGC CUGGCCCCGC C-3′ (SEQ ID NO:2), respectively.

The nucleotide sequences for the human miR-132 and miR-212 stem-loop precursor miRNAs are hsa-mir-132 MI0000449: 5′-CCGCCCCCGC GUCUCCAGGG CAACCGUGGC UUUCGAUUGU UACUGUGGGA ACUGGAGGUA ACAGUCUACA GCCAUGGUCG CCCCGCAGCA CGCCCACGCG C-3′ (SEQ ID NO:3) and hsa-mir-212 MI0000288: 5′-CGGGGCACCC CGCCCGGACA GCGCGCCGGC ACCUUGGCUC UAGAC GCUU ACUGCCCGGG CCGCCCUCAG UAACAGUCUC CAGUCACGGC CACCGACGCC UGGCCCCGCC-3′ (SEQ ID NO:4), respectively.

The nucleotide sequence for the mouse miR-132 and miR-212 stem-loop precursor miRNAs are mmu-mir-132 MI0000158: 5′-GGGCAACCGU GGCUUUCGAU UGUUACUGUG GGAACCGGAG GUAACAGUCU ACAGCCAUGG UCGCCC-3′ (SEQ ID NO:5) and mmu-mir-212 MI0000696: 5′-GGGCAGCGCG CCGGCACCUU GGCUCUAGAC UGCUUACUGC CCGGGCCGCC UUCAGUAACA GUCUCCAGUC ACGGCCACCG ACGCCUGGCC C-3′ (SEQ ID NO:6), respectively.

The mature miRNAs are the same across the three species. The nucleotide sequence for miR-132 is 5′-UAACAGUCUACAGCCAUGGU CG-3′ (SEQ ID NO:13) and the nucleotide sequence for miR-212 is 5′-UAACAGUCUCCAGUCACGGCC-3′ (SEQ ID NO:14). The nucleotide sequence for the miR-217 is 5′-UACUGCAUCAGGAACUGAUUGGAU-3′(SEQ ID NO:15) and the nucleotide sequence for miR-375 is 5′-UUUGUUCGUUCGGCUCGCGUGA-3′ (SEQ ID NO:16).

Therefore, in light of the discovery that miR-132 and miR-212 are expressed in β-cells in response to GLP-1, which effects an increase in cAMP, the present invention relates to the use of monitoring miR-132 and miR-212 expression to detect and evaluate islet engagement by GLP-1 receptor agonists and other agents that cause an increase in intracellular cAMP levels in β-cells. This is particularly useful in the context of longitudinal clinical trials, such as those designed for testing the durability of any single or combination therapy in type 2 diabetes populations. The present invention further relates to the use of monitoring miR-132 and/or miR-212 expression to monitor the efficacy of agents that effect an increase cAMP in β-cells.

The precise mechanism by which microRNAs are released from cells such as pancreatic beta-cell is not fully defined. The microRNAs present in plasma appear to come from many cell types including non-excitable ones (Wang K, et al. Proc. Natl. Acad. Sci. USA 106(11): 4402-7 (2009)). Our results indicate that the release of miR-132 and miR-212 is not regulated by ambient glucose levels and the amount released correlates very well with that produced inside the beta-cell. Furthermore, the expression levels and the function of many islet GPCRs signaling through cAMP, such as GLP-1R and GPR119, are not altered in diabetic condition. Agents that act on islet specific GPCRs (such as GLP-1R, GPR119, SSTR3, and SSTR5) will cause elevation of intracellular cAMP, which in turn stimulate the production and release of miR-132 and miR-212 into circulation. During this process, the amount of miR-132 and miR-212 released in response to the modulators of those islet GPCRs is determined by how many beta-cells available in the body. Thus, measuring the levels of miR-132 and/or miR-212 at various time points over time is particularly useful in the context of longitudinal clinical trials, such as those designed for testing the durability of any single or combination therapy in type 2 diabetic populations.

The methods herein are useful for determining the efficacy of agents that target the pancreatic islet β-cells and effect an increase in intracellular cAMP levels in the β-cells. The methods herein are useful for screening agents (drug candidates) that are to target the pancreatic islet β-cells and effect an increase in intracellular cAMP levels in the β-cells. The methods herein are useful for monitoring the progress of a treatment regime that uses an agent target the pancreatic islet β-cells and effect an increase in intracellular cAMP levels in the β-cells. Since diabetes is a progressive disease, therapies for managing the disease generally need to be changed or adjusted over time since a particular treatment in a patient may lose efficacy over time. Therefore, the methods of the present invention provide a rapid and noninvasive means for determining whether a particular therapy that targets the pancreatic islet β-cells and effects an increase in intracellular cAMP levels in the β-cells is still engaging the pancreatic islet β-cells.

Examples of agents that target the pancreatic islet β-cells and effect an increase in intracellular cAMP levels in the β-cells that are contemplated include but are not limited to glucagon-like peptide-1 (GLP-1) receptor agonists; glucagon peptide receptor agonists; agents that are both glucagon-like peptide-1 (GLP-1) receptor and a glucagon peptide receptor agonists; agents that are simultaneously glucagon-like peptide-1 (GLP-1) receptor agonists and glucagon peptide receptor antagonists; glucose-dependent insulinotropic polypeptide (GIP) receptor agonists; G-protein coupled receptor 119 (GPR-119) agonists; phosphodiesterase inhibitors, and dipeptidyl peptidase (DPP IV) inhibitors. GPR119 is also called RUP3 and SNORF 25.

In specific aspects, it is contemplated that the agent is selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, GPR-119 agonist, phosphodiesterase inhibitor, dipeptidyl peptidase (DPP IV) inhibitor, and combinations thereof.

In particular aspects, it is contemplated that the agent is an insulinotropic peptide selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, and combinations thereof.

Currently, most glucagon-like peptide-1 (GLP-1) receptor agonists, glucagon peptide receptor agonists, and glucose-dependent insulinotropic polypeptide (GIP) receptor agonists are either the receptor's cognate peptide or derivatives or analogs of the cognate peptide. Currently, delivery of these insulinotropic peptides to a subject or patient is achieved by injection. It would be desirable to identify small molecules that targets the pancreatic islet 13-cells and effects an increase in intracellular cAMP levels in the β-cells. These small molecules could be administered to a subject orally. The methods of the present invention provide a rapid and noninvasive means for confirming that a small molecule intended to target the pancreatic islet β-cells and effect an increase in intracellular cAMP levels in the β-cells is engaging the pancreatic islet β-cells.

The level of an miRNA in a sample can be measured using any technique that is suitable for detecting RNA expression levels in a biological sample. Suitable techniques (e.g., Northern blot analysis, reverse-transcription polymerase chain reaction (RT-PCR), quantitative or real-time RT-PCR, in situ hybridization) for determining RNA expression levels in a biological sample (e.g., serum plasma, cells, tissues) are well known to those of skill in the art.

Measuring miRNAs Using RT-PCR

In one aspect, determining the levels of miR-132 and/or mi-212 RNA transcripts can be accomplished determined by reverse transcription of miRNA followed by amplification of the reverse-transcribed transcripts by polymerase chain reaction (RT-PCR). The levels of the miRNA can be quantified in comparison with an internal standard, for example, the level of mRNA from a “housekeeping” gene present in the same sample. A suitable “housekeeping” gene for use as an internal standard includes, e.g., β-actin, myosin or glyceraldehyde-3-phosphate dehydrogenase (G3PDH). Methods for performing quantitative and semi-quantitative RT-PCR, and variations thereof, are well known to those of skill in the art. For example, U.S. Published Application No. 2005/0266418 to Chen describes a general RT-PCR method for detecting miRNAs, including miR-132 and miR-212.

For detecting miR-132, the RT-PCR method comprises obtaining total RNA from a bodily fluid sample from the subject and adding the total RNA to a reaction mixture comprising a linker probe having a stem, a loop, and a 3′ end sequence that base pairs with a 3′ end sequence of the miR-132, allowing the linker probe to hybridize the miR-132, and extending the linker probe to form an extension reaction product. The extension product is amplified to produce an amplification product in a polymerase chain reaction comprising a forward primer that hybridizes to the 5′ region of the miR-132 sequence in the extension or amplification product or a complementary sequence to the 5′ region of the miR-132 sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product. The detector probe produces a detectable signal during the course of the reaction when the total RNA from the bodily fluid sample contains miR-132. The absence of the detectable signal indicates the bodily fluid sample lacks miR-132. The linker probe is always a different molecule from the detector probe. In particular embodiments, the detector probe hybridizes to a nucleotide at or near the 3′ end region of the miR-132 sequence in the amplification product or hybridizes to a nucleotide at or near the 3′ end region of a complementary sequence of the miR-132 sequence in the amplification product.

For detecting miR-212, the RT-PCR method comprises obtaining total RNA from a bodily fluid sample from the subject and adding the total RNA to a reaction mixture comprising a linker probe having a stem, a loop, and a 3′ end sequence that base pairs with a 3′ end sequence of the miR-212, allowing the linker probe to hybridize the miR-212, and extending the linker probe to form an extension reaction product. The extension product is amplified to produce an amplification product in a polymerase chain reaction comprising a forward primer that hybridizes to the 5′ region of the miR-212 sequence in the extension or amplification product or a complementary sequence to the 5′ region of the miR-212 sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product. The detector probe produces a detectable signal during the course of the reaction when the total RNA from the bodily fluid sample contains miR-212. The absence of the detectable signal indicates the bodily fluid sample lacks miR-212. The linker probe is always a different molecule from the detector probe. In particular embodiments, the detector probe hybridizes to a nucleotide sequence at or near the 3′ end region of the miR-212 sequence in the amplification product or hybridizes to a nucleotide sequence at or near the 3′ end region of a complementary sequence of the miR-212 sequence in the amplification product.

For simultaneously detecting miR-132 and miR-212, the RT-PCR method comprises obtaining total RNA from a bodily fluid sample from the subject and adding the total RNA to a reaction mixture comprising a first linker probe having a stem, a loop, and a 3′ end sequence that base pairs with a 3′ end sequence of the miR-132 and a second linker probe having a stem, a loop, and a 3′ end sequence that base pairs with a 3′ end sequence of the miR-212, allowing the first and second linker probes to hybridize the appropriate miRNA, and extending the linker probes to form extension reaction products. The extension products are amplified to produce amplification products in a polymerase chain reaction comprising a first forward primer that hybridizes to the 5′ region of the miR-132 sequence in the extension or amplification product or a complementary sequence to the 5′ region of the miR-132 sequence in the extension or amplification product and a second forward primer that hybridizes to the 5′ region of the miR-212 sequence in the extension or amplification product or a complementary sequence to the 5′ region of the miR-212 sequence in the extension or amplification product, a reverse primer that hybridizes to the linker probe sequence in the extension or amplification product or a complementary sequence to the linker probe sequence in the extension or amplification product, and a first detector probe that hybridizes to a nucleotide sequence at or near the 3′ end region of the miR-132 sequence in the amplification product or hybridizes to a nucleotide sequence at or near the 3′ end region of a complementary sequence of the miR-132 sequence in the amplification product and a second detector probe that hybridizes to a nucleotide sequence at or near the 3′ end region of the miR-212 sequence in the amplification product or hybridizes to a nucleotide sequence at or near the 3′ end region of a complementary sequence of the miR-212 sequence in the amplification product. The first detector probe produces a detectable signal over time during the course of the reaction that is distinguishable from the detectable signal that is produced by the second detector probe over time during the course of the reaction.

As used herein, the term “linker probe” refers to a molecule comprising a stem, a loop, and a 3′ end sequence that base pairs with a 3′ end sequence of the miRNA. It will be appreciated that the linker probes, as well as the primers herein, can be comprised of ribonucleotides, deoxyribonucleotides, modified ribonucleotides, modified deoxyribonucleotides, modified phosphate-sugar-backbone oligonucleotides, nucleotide analogs, or combinations thereof. For examples of various nucleotide analogs, etc, See Fasman, 1989, Practical Handbook of Biochemistry and Molecular Biology, pp. 385-394, CRC Press, Boca Raton, Fla.; Loakes, Nucl. Acid Res. 29: 2437-2447 (2001); and Pellestor et al., Int. J. Mol. Med. 13(4): 521-5 (2004).

The 3′ end sequence of the linker probe that base pairs with a 3′ end sequence of the miRNA is located downstream from the stem of the linker probe. Generally, the 3′ end sequence of the linker probe is between 6 and 8 nucleotides long. In some embodiments, the 3′ end sequence is 7 nucleotides long. It will be appreciated that routine experimentation can produce other lengths and that sequences that are longer than 8 nucleotides or shorter than 6 nucleotides can also be used. Generally, the 3′-most nucleotides of the 3′ end sequence of the linker probe should have minimal complementarity overlap, or no overlap at all, with the 3′ nucleotides of the forward primer: it will be appreciated that overlap in these regions can produce undesired primer dimer amplification products in subsequent amplification reactions. In some embodiments, the overlap between the 3′-most nucleotides of the 3′ end sequence of the linker probe and the 3′ nucleotides of the forward primer is 0, 1, 2, or 3 nucleotides. In some embodiments, greater than 3 nucleotides can be complementary between the 31-most nucleotides of the 3′ end sequence of the linker probe and the 3′ nucleotides of the forward primer but generally such scenarios will be accompanied by additional non-complementary nucleotides interspersed therein. In some embodiments, modified bases such as locked nucleic acids (LNA) can be used in the 3′ end sequence of the linker probe to increase the Tm of the linker probe (See for example Petersen et al., Trends Biochem. 21: 74-81 (2003)). In some embodiments, modifications including but not limited to LNAs and universal bases can improve reverse transcription specificity and potentially enhance detection specificity.

As used herein, the term “stem” refers to the double stranded region of the linker probe that is between the 3′ end sequence of the linker probe and the loop. Generally, the stem is between 6 and 20 nucleotides long (that is, 6-20 complementary pairs of nucleotides, for a total of 12-40 distinct nucleotides). In some embodiments, the stem is 8-14 nucleotides long. As a general matter, in those embodiments in which a portion of the detector probe is encoded in the stem, the stem can be longer. In those embodiments in which a portion of the detector probe is not encoded in the stem, the stem can be shorter. Those in the art will appreciate that stems shorter that 6 nucleotides and longer than 20 nucleotides can be identified in the course of routine methodology and without undue experimentation, and that such shorter and longer stems are contemplated by the present teachings. In some embodiments, the stem can comprise an identifying portion.

As used herein, the term “loop” refers to a region of the linker probe that is located between the two complementary strands of the stem. Typically, the loop comprises single stranded nucleotides, though other moieties modified DNA or RNA, carbon spacers such as C₁₈, and/or PEG (polyethylene glycol) are also possible. Generally, the loop is between 4 and 20 nucleotides long. In some embodiments, the loop is between 14 and 18 nucleotides long. In some embodiments, the loop is 16 nucleotides long. As a general matter, in those embodiments in which a reverse primer is encoded in the loop, the loop can generally be longer. In those embodiments in which the reverse primer corresponds to both the target polynucleotide as well as the loop, the loop can generally be shorter. Those in the art will appreciate that loops shorter that 4 nucleotides and longer than 20 nucleotides can be identified in the course of routine methodology and without undue experimentation, and that such shorter and longer loops are contemplated by the present teachings. In some embodiments, the loop can comprise an identifying portion.

An example of an RT linker probe that can be used in the method herein to detect miR-132 has the nucleotide sequence 5′-GTCGTATCCAGTGCAGGGTCCGAGGTATT CGCACTGGATACGACCGACCA-3′ (SEQ ID NO:17). An example of a linker probe that can be used in the method herein to detect miR-212 has the nucleotide sequence 5′-GTCGTATCCAGTGCAGGGTCCGAGGTAGGCGCACTGGATACGACGGCCGT-3′ (SEQ ID NO:21).

As used herein, the term “extension reaction” refers to an elongation reaction in which the 3′ end sequence of the linker probe is extended to form an extension reaction product comprising a strand complementary to the target polynucleotide. In some embodiments, the extension reaction is a reverse transcription reaction comprising a reverse transcriptase. In some embodiments, the extension reaction is a reverse transcription reaction comprising a polymerase derived from a Eubacteria. In some embodiments, the extension reaction can comprise rTth polymerase, for example as commercially available from Applied Biosystems catalog number N808-0192, and N808-0098. Polymerases that also comprise reverse transcription properties can allow for embodiments that comprise a first reverse transcription reaction followed immediately thereafter by an amplification reaction, thereby allowing for the consolidation of two reactions in essentially a single reaction.

As used herein, the term “forward primer” refers to a primer that comprises an extension reaction product portion and a tail portion. The extension reaction product portion of the forward primer hybridizes to the extension reaction product. Generally, the extension reaction product portion of the forward primer is between 9 and 19 nucleotides in length. In some embodiments, the extension reaction product portion of the forward primer is 16 nucleotides. The tail portion is located upstream from the extension reaction product portion, and is not complementary with the extension reaction product; after a round of amplification however, the tail portion can hybridize to complementary sequence of amplification products. Generally, the tail portion of the forward primer is between 5-8 nucleotides long. In some embodiments, the tail portion of the forward primer is 6 nucleotides long.

An example of a forward primer that can be used in the method herein to detect miR-132 has the nucleotide sequence 5′-GCCGCTAACAGTCTACAGCCAT-3′ (SEQ ID NO:18). An example of a linker probe that can be used in the method herein to detect miR-212 has the nucleotide sequence 5′-GCCGCTAACAGTCTCCAGTCA-3′ (SEQ ID NO:22).

As used herein, the term “reverse primer” refers to a primer that when extended forms a strand complementary to the target polynucleotide. In some embodiments, the reverse primer corresponds with a region of the loop of the linker probe. Following the extension reaction, the forward primer can be extended to form a second strand product. The reverse primer hybridizes with this second strand product, and can be extended to continue the amplification reaction. In some embodiments, the reverse primer corresponds with a region of the loop of the linker probe, a region of the stem of the linker probe, a region of the target polynucleotide, or combinations thereof. Generally, the reverse primer is between 13-16 nucleotides long. In some embodiments the reverse primer is 14 nucleotides long. In some embodiments, the reverse primer can further comprise a non-complementary tail region, though such a tail is not required.

An example of a reverse primer that can be used in the method herein to detect miR-132 or mR-212 has the nucleotide sequence 5′-GTGCAGGGTCCGAGGT-3″ (SEQ ID NO:19). It should be noted that this primer is a universal primer that corresponds to a sequence spanning the stem and loop region of the linker probe; therefore, it can be used in the reactions to detect either miR-132 or miR-212 or any other miRNA that includes same nucleotide sequence.

The term “upstream” as used herein takes on its customary meaning in molecular biology, and refers to the location of a region of a polynucleotide that is on the 5′ side of a “downstream” region. Correspondingly, the term “downstream” refers to the location of a region of a polynucleotide that is on the 3′ side of an “upstream” region.

As used herein, the term “hybridization” refers to the complementary base-pairing interaction of one nucleic acid with another nucleic acid that results in formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing detector probes and primers to complementary sequences are well known, e.g., as described in Nucleic Acid Hybridization, A Practical Approach, Haines & Higgins, eds., IRL Press, Washington, D.C. (1985) and Wetmur & Davidson, Mol. Biol. 31: 349 (1968). In general, whether such annealing takes place is influenced by, among other things, the length of the polynucleotides and the complementary, the pH, the temperature, the presence of monovalent and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the single stranded nucleic acids of the present teachings. However, if the number of base pair mismatches is so great that no hybridization can occur under minimally stringent conditions then the sequence is generally not a complementary target sequence.

As used herein, the term “amplifying” refers to any means by which at least a part of a target polynucleotide, target polynucleotide surrogate, or combinations thereof, is reproduced, typically in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34: 501-507 (1996); The Nucleic Acid Protocols Handbook, Rapley, ed., Humana Press, Totowa, N.J. (2002); Abramson et al., Curr Opin Biotechnol. 1993 February; 4(1): 41-47, U.S. Pat. No. 6,027,998; U.S. Pat. No. 6,605,451, Barany et al., PCT Application Pub. No. WO 97/31256; Wenz et al., PCT Application Pub. No. WO 01/92579; Day et al., Genomics 29: 152-162 (1995), Ehrlich et al., Science 252: 1643-50 (1991); Innis et al., PCR Protocols: A Guide to Methods and Applications, Academic Press (1990); Favis et al., Nature Biotechnology 18: 561-64 (2000); and Rabenau et al., Infection 28:97-102 (2000); Barany, Proc. Natl. Acad. Sci. USA 88: 188-93 (1991); Bi & Sambrook, Nucl. Acids Res. 25:2924-2951 (1997); Zirvi et al., Nucl. Acid Res. 27: e40i-viii (1999); Dean et al., Proc Natl. Acad. Sci. USA 99: 5261-66 (2002); Barany & Gelfand, Gene 109: 1-11 (1991); Walker et al., Nucl. Acid Res. 20: 1691-96 (1992); Polstra et al., BMC Inf. Dis. 2: 18 (2002); Lage et al., Genome Res. 13: 294-307 (2003), Landegren et al., Science 241: 1077-80 (1988), Demidov, Expert. Rev. Mol. Diagn. 2: 542-8 (2002), Cook et al., J. Microbiol. Methods 53: 165-74 (2003); Schweitzer et al., Curr. Opin. Biotechnol. 12: 21-7 (2001), U.S. Pat. No. 5,830,711, U.S. Pat. No. 6,027,889, U.S. Pat. No. 5,686,243, PCT Application Pub. No. WO00/56927A3, and PCT Application Pub. No. WO98/03673A1. In some embodiments, newly-formed nucleic acid duplexes are not initially denatured, but are used in their double-stranded form in one or more subsequent steps. An extension reaction is an amplifying technique that comprises elongating a linker probe that is annealed to a template in the 5′ to 3′ direction using an amplifying means such as a polymerase and/or reverse transcriptase. According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed linker probe, to generate a complementary strand. In some embodiments, the polymerase used for extension lacks or substantially lacks 5′ exonuclease activity. In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, amplification can be achieved in a self-contained integrated approach comprising sample preparation and detection, as described for example in U.S. Pat. Nos. 6,153,425 and 6,649,378.

As used herein, the term “detector probe” refers to a molecule used in an amplification reaction, typically for quantitative or real-time PCR analysis, as well as end-point analysis. Such detector probes can be used to monitor the amplification of the target polynucleotide. In some embodiments, detector probes present in an amplification reaction are suitable for monitoring the amount of amplicon(s) produced as a function of time. Such detector probes include, but are not limited to, the 5′-exonuclease assay TAQMAN probes described herein (See also U.S. Pat. No. 5,538,848), various stem-loop molecular beacons (See e.g., U.S. Pat. Nos. 6,103,476 and 5,925,517 and Tyagi & Kramer, Nat. Biotechnol. 14: 303-308 (1996)), stemless or linear beacons (see, e.g., PCT Application Pub. No. WO 99/21881), PNA Molecular Beacons (See, e.g., U.S. Pat. Nos. 6,355,421 and 6,593,091), linear PNA beacons (See, e.g., Kubista et al., SPIE 4264: 53-58 (2001)), non-FRET probes (See, e.g., U.S. Pat. No. 6,150,097), SUNRISE./AMPLIFLUOR probes (U.S. Pat. No. 6,548,250), stem-loop and duplex SCORPION probes (Solinas et al., Nucl. Acids Res. 29: E96 (2001) and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB ECLIPSE probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., Methods 25: 463-471 (2001); Whitcombe et al., Nat. Biotechnol. 17: 804-807 (1999); Isacsson et al., Molec. Cell. Probes. 14: 321-328 (2000); Svanvik et al., Anal. Biochem. 281: 26-35 (2000); Wolffs et al., Biotech. 766: 769-771 (2001); Tsourkas et al., Nucl. Acids Res. 30: 4208-4215 (2002); Riccelli et al., Nucl. Acids Res. 30: 4088-4093 (2002); Maxwell et al., J. Am. Chem. Soc. 124: 9606-9612 (2002); Broude et al., Trends Biotechnol, 20: 249-56 (2002); Huang et al., Chem. Res. Toxicol. 15: 118-126 (2002); and Yu et al., J. Am. Chem. Soc 14: 11155-11161 (2001).

Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe and a quencher is on the other probe, wherein hybridization of the two probes together on a target quenches the signal or wherein hybridization on the target alters the signal signature via a change in fluorescence. Detector probes can also comprise sulfonate derivatives of fluorescenin dyes with SO₃ instead of the carboxylate group, phosphoramidite forms of fluorescein, phosphoramidite forms of CY 5 (commercially available for example from Amersham). In some embodiments, interchelating labels are used such as ethidium bromide, SYBR Green I (Molecular Probes), and PICOGREEN (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, the detector probes have a Tm of 63-69° C., though it will be appreciated that experimentation can result in detector probes with other Tms. In some embodiments, probes can further comprise various modifications such as a minor groove binder (MGB) (See for example U.S. Pat. No. 6,486,308) to further provide desirable thermodynamic characteristics. In some embodiments, detector probes can correspond to identifying portions or identifying portion complements.

An example of a detector probe that can be used in the method herein to detect miR-132 has the nucleotide sequence 5′-TGGATACGACCGACCAT-3′ (SEQ ID NO:20). An example of a linker probe that can be used in the method herein to detect miR-212 has the nucleotide sequence 5′-TGGATACGAC GGCCGTG-3′ (SEQ ID NO:23). The detector probes further can further be conjugated at the 5′ end to a fluorophore, e.g., 6-carboxyfluorescein (6-FAM). The 3′ end of the detector probes can be conjugated to a quencher that quenches fluorescence of the fluorophore when it is in close proximity as when conjugated to the detector probe. A quencher suitable for quenching expression of 6-FAM is tetramethylrhodamine (TAMRA). In particular aspects of the detector probe, the 3′ end of the detector probe is bound to a minor groove binder (MGB) ligand, which is conjugated to the quencher.

As used herein, the term “detection” refers to any of a variety of ways of determining the presence and/or quantity and/or identity of a target polynucleotide. In some embodiments employing a donor moiety and signal moiety, one may use certain energy-transfer fluorescent dyes. Certain nonlimiting exemplary pairs of donors (donor moieties) and acceptors (signal moieties) are illustrated, e.g., in U.S. Pat. Nos. 5,863,727; 5,800,996; and 5,945,526. Use of some combinations of a donor and an acceptor have been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, VIC, LIZ, TAMRA, 5-FAM, 6-FAM, (all available from Applied Biosystems, Foster City, Calif.) and Texas Red (Molecular Probes). In some embodiments, the amount of detector probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator. According to some embodiments, one can employ an internal standard to quantify the amplification product indicated by the fluorescent signal (See, e.g., U.S. Pat. No. 5,736,333). Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI PRISM 7700 Sequence Detection System (Applied Biosystems, Foster City, Calif.), the ABI GENEAMP 5700 Sequence Detection System (Applied Biosystems, Foster City, Calif.), the ABI GENEAMP 7300 Sequence Detection System (Applied Biosystems, Foster City, Calif.), and the ABI GENEAMP 7500 Sequence Detection System (Applied Biosystems).

In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product. In some embodiments, combined thermal cycling and fluorescence detecting devices can be used for precise quantification of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification. In some embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target polynucleotide. As used herein, determining the presence of a target can comprise identifying it, as well as optionally quantifying it. In some embodiments, the amplification products can be scored as positive or negative as soon as a given number of cycles is complete. In some embodiments, the results may be transmitted electronically directly to a database and tabulated. Thus, in some embodiments, large numbers of samples can be processed and analyzed with less time and labor when such an instrument is used.

In some embodiments, different detector probes can distinguish between different target polynucleotides. A non-limiting example of such a probe is a 5′-nuclease fluorescent probe, such as a TAQMAN probe molecule, wherein a fluorescent molecule is attached to a fluorescence-quenching molecule through an oligonucleotide link element. In some embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WL_{A} and WL_{B}) and that are specific to two different stem regions of two different extension reaction products (A′ and B′, respectively). Amplification product A′ is formed if target nucleic acid sequence A is in the sample, and amplification product B′ is formed if target nucleic acid sequence B is in the sample. In some embodiments, amplification product A′ and/or B′ may form even if the appropriate target nucleic acid sequence is not in the sample, but such occurs to a measurably lesser extent than when the appropriate target nucleic acid sequence is in the sample. After amplification, one can determine which specific target nucleic acid sequences are present in the sample based on the wavelength of signal detected and their intensity. Thus, if an appropriate detectable signal value of only wavelength WL_{A} is detected, one would know that the sample includes target nucleic acid sequence A, but not target nucleic acid sequence B. If an appropriate detectable signal value of both wavelengths WL_{A} and WL_{B} are detected, one would know that the sample includes both target nucleic acid sequence A and target nucleic acid sequence B. In some embodiments, detection can occur through any of a variety of mobility dependent analytical techniques based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques, and the like. In some embodiments, mobility probes can be hybridized to amplification products, and the identity of the target polynucleotide determined via a mobility dependent analysis technique of the eluted mobility probes, as described for example in PCT Application Pub. Nos. WO2004/46344 to Rosenblum et al., and WO01/92579 to Wenz et al. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (See also Gerry et al., J. Mol. Biol. 292: 251-62 (1999); De Bellis et al., Minerva Biotec. 14: 247-52 (2002); and Stears et al., Nat. Med. 9: 14045 (2003)). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the detecting the detector probe.

Measuring miRNAs Using Northern Blot Analysis

In another aspect embodiment, the level of miR-132 and/or miR-212 is detected using Northern blot analysis. For example, total cellular RNA can be purified from cells or serum plasma by homogenization in the presence of nucleic acid extraction buffer followed by centrifugation. Nucleic acids are precipitated and DNA is removed by treatment with DNase and precipitation. The RNA molecules are then separated by gel electrophoresis on agarose gels according to standard techniques and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes (e.g., DNA probes, RNA probes) for Northern blot hybridization of a given miR-132 or miR-212 include, but are not limited to, probes having at least about 70%, 75%, 80%, 85%, 90%, 95%, 98% or 99% complementarity to miR-132 or miR-212, as well as probes that have complete complementarity to the miRNA. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as ³H, ³²P, ³³P, ¹⁴C, or ³⁵S; a heavy metal; a ligand capable of functioning as a specific binding pair member for a labeled ligand (e.g., biotin, avidin or an antibody); a fluorescent molecule; a chemiluminescent molecule; an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of miRNA levels. Using another approach, miRNA levels can be quantified by computerized imaging systems, such as the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin and antibodies (e.g., anti-biotin antibodies) coupled to fluorescent dyes or enzymes that produce color reactions.

Non-Invasive Measurement of microRNAs Using Various Imaging Modalities

While endogenous miRNA levels can be evaluated by several methods, including northern blotting, TAQMAN quantitative PCR, and DNA chip analyses after the extraction of total RNAs from biopsy and bodily fluid samples, these methods are time-consuming and cannot provide real-time information of miRNA changes in pancreatic β-cell tissues in a noninvasive manner.

Recently, Kim et al. (Mol. Imaging Biol. 11(2): 71-78 (2009) Epub 2008 Nov. 22) reported a novel bioluminescence imaging (BLI) strategy to evaluate and visualize the expression levels of several miRNAs using specific reporter vectors. For instance, in a miR-221 specific reporter vector, three repeat perfect complementary target sequences (3×PT) of mature miR-221 were inserted right after stop codon of the Gaussian luciferase (Glue) gene. An increase of endogenous miR-221 is expected to bind with the transcript of this vector, thus suppressing the translation of Glue or degrading the transcript, resulting in a decrease of BLI signal. In this way, endogenous miRNA expression levels can be indirectly visualized and monitored non-invasively. This approach has been successfully used to monitor expression levels of miR-221, miR-9 and miR-23a (Kim et al., FEBS J. 276: 2165-2174 (2009). The method can used to monitor expression levels of miR-132 and miR-212 in pancreatic tissue.

In addition to the BLI modality (with luciferase reporter), the more clinically relevant positron emission tomography (PET) or magnetic resonance imaging (MRI) reporters such as thymidine kinase, sodium iodide symporter, or ferritin can be used to measure microRNAs (Niu & Chen, Mol. Imaging Biol. 11(2): 61-63 (2009). Fluorescent dyes or radioisotopes labeled oligonucleotides for targeting miRNAs may also be used to image or quantify endogenous miRNA level after systemic administration. The future development in the molecular imaging of miRNAs will not only greatly advance our understanding of physiological and pathophysiological roles of miRNAs but also enhance our ability to monitor the engagement of novel therapeutic agents with β-cell targets in clinical studies.

The primers and probes herein can be provided in kits to be used for monitoring pancreatic islet β-cell engagement by agents intended to target pancreatic islet β-cells and effect an increase in intracellular cAMP levels.

The following examples are intended to promote a further understanding of the present invention.

### Example 1

Glucagon like peptide-1 (GLP-1) exerts pleiotropic effects on pancreatic β-cell function. GLP-1 potentiates glucose-dependent insulin secretion (GDIS) in pancreatic β-cells. Chronic administration of GLP-1 also promotes insulin synthesis as well as β-cell proliferation and neogenesis, at least in animal models. The detailed underlying mechanism remains to be fully understood. In this example, the expression levels of microRNAs in INS-1 832/3 cell, a clonal rat insulinoma cell line which exhibits robust glucose-dependent insulin secretion, was profiled. The expression levels of 250 microRNAs in INS-1 832/3 cell cultured in the presence or absence of 50 nM GLP-1 for 24 hours were compared using quantitative reverse transcription polymerase chain reaction (RT-PCR) method.

INS-1 832/3 and 832/13, two clonal rat insulinoma cell lines, which exhibit robust glucose-dependent insulin secretion, were obtained from Dr. Christopher Newgard at Duke University (Newgard et al., Diabetes Suppl. 3: S389-93, (2002)) and maintained in RPMI 1640 with 10% FBS and 11 mM glucose. For GLP-1 treatment, GLP-1 (7-37) amide (ABCHEM) was added in the media in the presence of 16 mM glucose for 24 hours.

For microRNA profiling, total RNA was extracted from INS-1 832/3 cells with TRIZOL reagent (Invitrogen, Carlsbad, Calif.). A total of 250 miRNA species were determined by SYBR green quantitative PCR (qPCR) method at Rossetta Inpharmatics (Seattle, Wash.). Quantitative PCR is a method used to detect relative or absolute gene expression level. All qPCR involves the use of fluorescence to detect the threshold cycle (Ct) during PCR when the level of fluorescence gives signal over the background and is in the linear portion of the amplified curve. This Ct value is responsible for the accurate quantization of qPCR. In this example, the Ct value data was calculated into copy number per 10 pg total RNA using the standard curve method. See for example, Raymon et al. (RNA 11: 1737-1744 (2005). Data of three replicate using cells at different passages was analyzed using the ROSETTA RESOLVER system, version 7.1 (Rosetta Biosoftware, Seattle, Wash.).

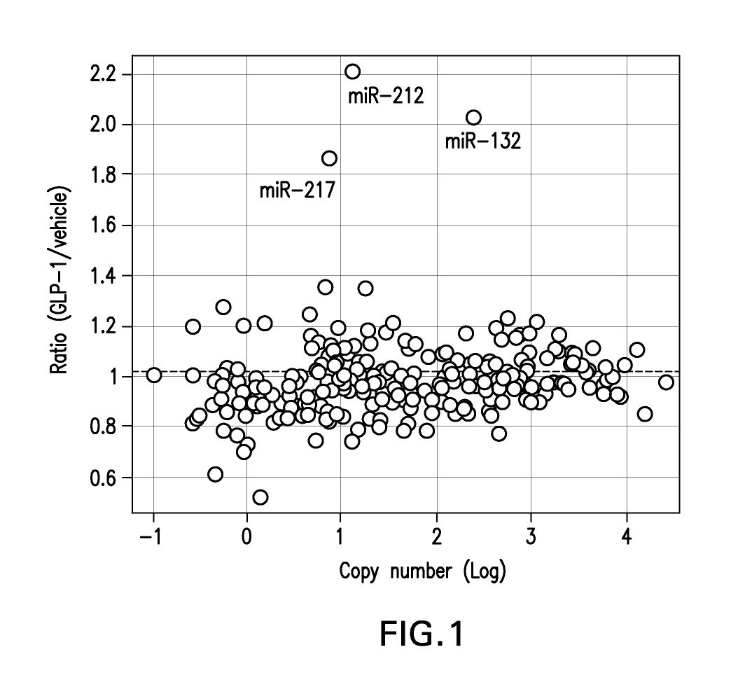

Expression of 219 miRNAs was detectable in the INS-1 832/3 cell line and 147 of the miRNAs (67%) were in amounts greater than 10 copies per 10 pg total RNA. The average copy numbers versus fold of induction by GLP-1 is shown in FIG. 1. We observed no significant changes in the expression of the majority of miRNAs analyzed, including a number of miRNAs previously known to be involved in the regulation of β-cell function (such as miR-375, miR-9 and miR-124a (Poy et al., Nature 432: 226-230, (2004); Plaisance et al., J. Biol. Chem. 281: 26932-26942 (2006); Baroukh et al., J. Biol. Chem. 282: 19575-19588 (2007)). However, three miRNAs, miR-132 and miR-212 and miR-217 were found to be up-regulated 1.8 to 2.2 fold (FIG. 1). Their average copy numbers were 246, 13 and 7 per 10 pg total RNA, respectively. The changes in miR-132 and miR-212 were consistent for all three replicates (i.e., three experiments on three separate days), whereas up-regulation of miR-217 was only observed in one of the three replicate. Subsequent TAQMAN analyses also confirmed the induction of miR-132 and miR-212 but not miR-217.

### Example 2

GLP-1-mediated regulation of miR-132 and miR-212 in INS-1 832/3 cells and rat pancreatic islets was confirmed with TAQMAN analysis.

INS-1 832/3 and 832/13 cells were maintained in RPMI 1640 with 10% FBS and 11 mM glucose. For GLP-1 treatment, GLP-1 (7-37) amide (ABCHEM) was added in the media in the presence of 16 mM glucose for 24 hours. Total RNA was extracted with TRIZOL as in Example 1. Rat islet isolation and treatment was as follows. Pancreatic islets of Langerhans were isolated from the pancreas of normal Sprague-Dawley rats (Charles River, Ind.) by collagenase digestion and discontinuous Ficoll gradient separation (15). Islets were cultured for 2 hours in RPMI 1640 medium with 11 mM glucose for recovery from the isolation process. Then 100-200 islets were treated with 50 nM GLP-1 in the media with 11 mM glucose for 24 hours. RNA was extracted with TRIZOL as in Example 1 for quantification of microRNA species.

TAQMAN real-time quantitative RT-PCR analyses confirmed the induction of miR-132 and miR-212. Fluorogenic TAQMAN detector probes specific for miR-132 (Cat# TM457), miR-212 (Cat# TM515), miR-375 (Cat#TM564), and miR-217 (Cat#1133) were purchased from Applied Biosystems (Foster City, Calif.). The TAQMAN RT-PCR reactions were performed according to the manufacturer's instructions.

Relative miRNA levels for the miRNAs of interest were determined by real-time reverse transcription reaction using the ABI PRISM 7900 Sequence Detection System from Applied Biosystems (Foster City, Calif.) according to the manufacturer's protocol. Briefly, 10 ng of total RNA was mixed with 1 U MultiScribe Reverse Transcriptase, 0.25 U RNase Inhibitor, 3 μL hairpin-looped miRNA-specific RT primer, 1 mM dNTPs and 1× Reverse Transcription Buffer in a total volume of 15 μL. The mixture was incubated at 16° C. for 30 minutes, 42° C. for another 30 minutes, and the reaction was stopped by heating to 85° C. for 5 minutes. Real time PCR reaction was set up in 20 μL volume with 1.33 μL first strand cDNA, 1× TAQMAN MicroRNA Assay Mix and 1× TAQMAN Universal PCR Master Mix. After activation of the AMPLITAQ Gold DNA polymerase at 95° C. for 10 minutes, 40 cycles of two-step PCR were run (95° C. for 15 seconds and 60° C. for 60 seconds). Data were collected and analyzed with SDS v.2.2.2 software (Applied Biosystems). 4.5S RNA or U6 probe (Applied Biosystems) was used as a reference to determine the relative abundance of each miRNA in different samples. TAQMAN RT-PCR was performed according the manufacturer's instructions.

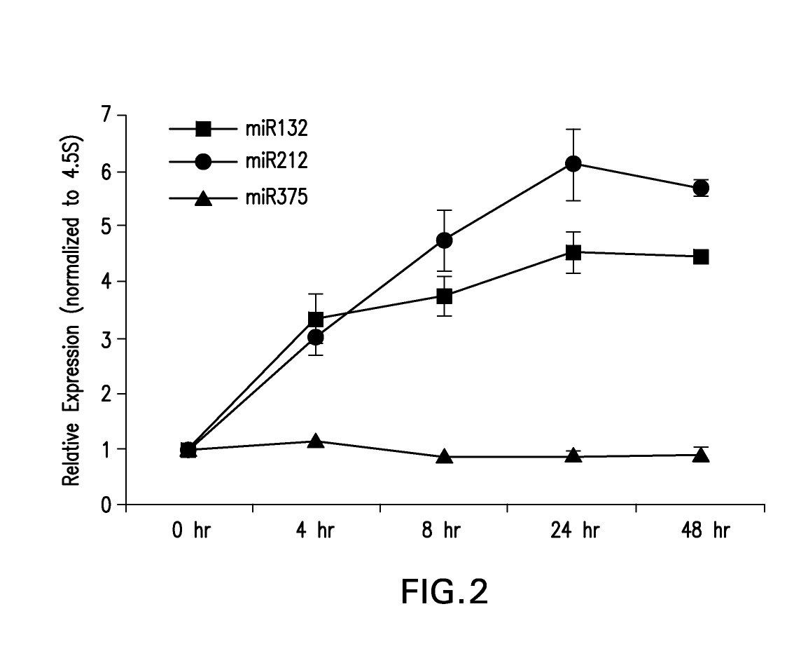

The absolute miRNA levels were determined by reverse transcription reactions in GeneAmp PCR system 9700 followed by amplification using the ABI PRISM 7900HT Sequence Detection System from Applied Biosystems (Foster City, Calif.) through 40 cycles. 4.5S RNA (H) probe (Cat#TM1716) or U6 (Cat#TM1973) was used as reference to determine the relative abundance of each miRNA in different samples. The induction of miR-132 and miR-212 expression by GLP-1 started around 4 hour post-GLP-1 treatment, peaked at 24 hours and was sustained up to 48 hours (FIG. 2). This time course of GLP-1-mediated regulation suggests that these two miRNAs are likely involved in chronic regulation of β-cell function, rather than the acute GDIS response itself.

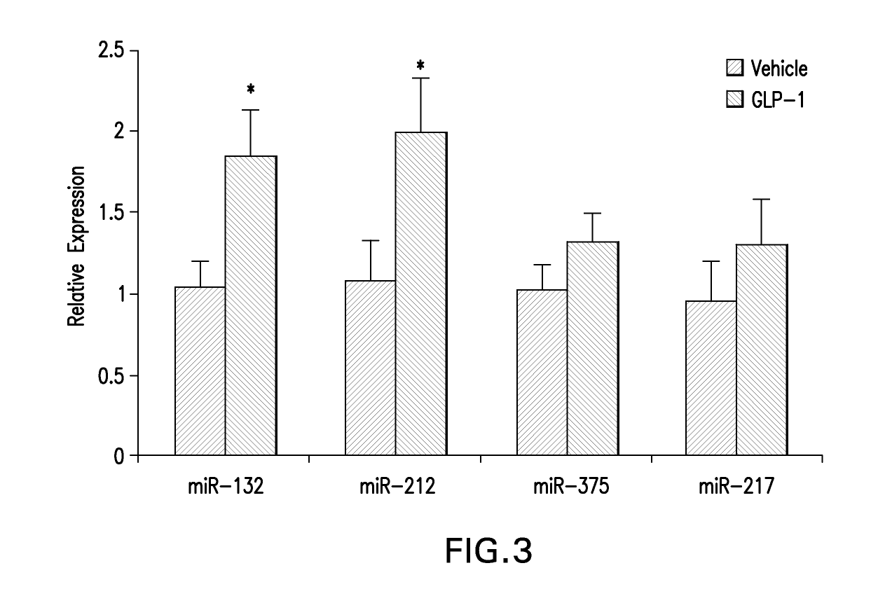

To verify that GLP-1 mediated miR-212 and miR-132 expression changes are of biological relevance, we analyzed their expression in freshly isolated rat islets treated with GLP-1. Pancreatic islets of Langerhans were isolated from the pancreas of normal Sprague-Dawley rats (Charles River, Ind.) by collagenase digestion and discontinuous Ficoll gradient separation. Islets were cultured for two hours in RPMI 1640 medium with 11 mM glucose for recovery from the isolation process. Then 100 to 200 islets were treated with 50 nM GLP-1 in the media with 11 mM glucose for 24 hours. RNA was extracted with TRIZOL reagent/ethanol precipitation followed by quantification of miRNA species. After 24 hour treatment, the expression levels of both miR-132 and miR-212 were significantly increased by 2 fold (FIG. 3). Neither miR-375 nor miR-217 was significantly regulated by GLP-1 in rat islets. The regulation of miR-132 and miR-212 expression by GLP-1 was confirmed in rat pancreatic islets.

### Example 3

This example shows that GLP-1 promotes the release of miR-132 and miR-212 from INS-1 832/3 cells in cell culture and the released miRNAs are detectable using quantitative real-time RT-PCR.

INS1 832/3 cells were treated with 50 nM GLP-1 (7-37) amide (BACHEM) in RPMI 1640 with 10% FBS and 11 mM glucose in 24-well plates for 4, 8, 16, and 24 hours. Cell culture media was collected, 100 μL out of 1 mL media per sample was used for RNA extraction following the protocol for plasma samples (Mitchell et al., Proc. Natl. Acad. Sci. USA 105: 10513-10518). Briefly, RNA isolation was performed using the mirVana PARIS kit following the manufacturer's protocol (Ambion), except that samples were extracted twice with acid phenol-chloroform. The average of about 80 μL of eluate was recovered from each extraction. A fixed volume of 1.67 μL of the RNA eluate was used as input into the reverse transcription reaction. RNA was reverse transcribed using the TAQMAN miRNA Reverse Transcription Kit and miRNA-specific stem-loop primers in 5 μL volume (Applied BioSystems). About 2.25 μL of diluted PCR product (combining 5 μL RT product with 28.9 μL H₂O) was added to 2.75 μL of PCR assay reagents to generate a PCR of 5 μL of total volume. The relative miRNA levels were determined by real-time RT-PCR using the ABI PRISM 7900 Sequence Detection System from Applied Biosystems (Foster City, Calif.) through 40 cycles as described in Example 2. TAQMAN reagents were purchased from Applied Biosystems (Foster City, Calif.). TAQMAN analysis for miR-132, miR-212, miR-375, and U6 RNA were performed and microRNA levels were normalized to U6 RNA levels.

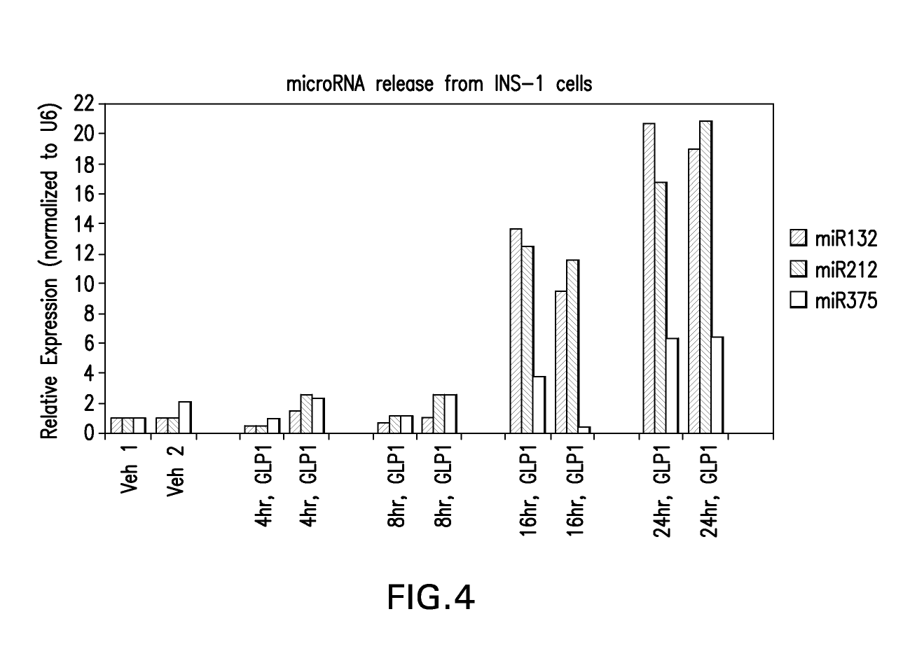

Compared to vehicle treatment, miR-132 and miR-212 expression in the medium remained unchanged after 4 or 8 hours GLP-1 treatment. At 16 and 24 hour post-treatment, miR-132 and miR-212 levels were significantly increased by 10 to 20 fold (FIG. 4) suggesting GLP-1 promotes the release of these microRNAs from INS-1 832/3 cells.

### Example 4

Because GLP-1 promote the release of miR-132 and miR-212 in the INS-1 832/3 cell and mouse islets, activation of the GLP-1 receptor (GLP-1R) in viva by, for example, administration of a GLP-1 is expected to cause a surge in the release of miR-132 and miR-212 from the pancreatic islets with the subsequent elevation of plasma levels of miR-132 and miR-212. The elevated levels of miR-132 and miR-212 serve as circulating biomarkers of the engagement of the target (GLP-1R) by a GLP-1R agonist (e.g., GLP-1, oxyntomodulin, and the like). To demonstrate this effect, we measured the plasma levels of miR-132 and miR-212 in lean mice treated with a long-acting GLP-1 receptor agonist. As shown in this example, treatment with the long-acting GLP-1 receptor agonist effected an increase in plasma levels of miR-132 and miR-212 in normal mice.

The plasma levels of miR-132 and miR-212 were measured in lean mice treated with an oxyntomodulin (OXM) derivative (a long-acting GLP-1 receptor agonist disclosed in Published International Application No. WO2007/100535) before and then 24-hours and 48-hours after a single injection of the OXM derivative. Groups of 16 weeks old mice were dosed with 30 mpk OXM derivative (s.c.) or vehicle (3% mannitol, 75 mM NaCl, 20 mM sodium acetate, pH 5) and approximately 50 μL blood each were collected into EDTA tubes before and 24 hours and 48 hours post-dosing. All the blood samples were immediately placed on ice and plasma were prepared within one hour and stored at −80° C. RNA extraction and TAQMAN RT-PCR analysis were as described in Example 3. 50 μL aliquots of the plasma samples were thawed on ice and 2× Denaturing Solution (Ambion) was added. To allow for normalization for sample variations, three C. elegans miRNAs, cel-miR39, cel-miR54, and cel-miR-238, were added at a final concentration of 20, 200, and 2000 fM (sequence information according to miRBase). miR132, miR-212, and miR-375 expression levels were normalized to spiked-in cel-miR39.

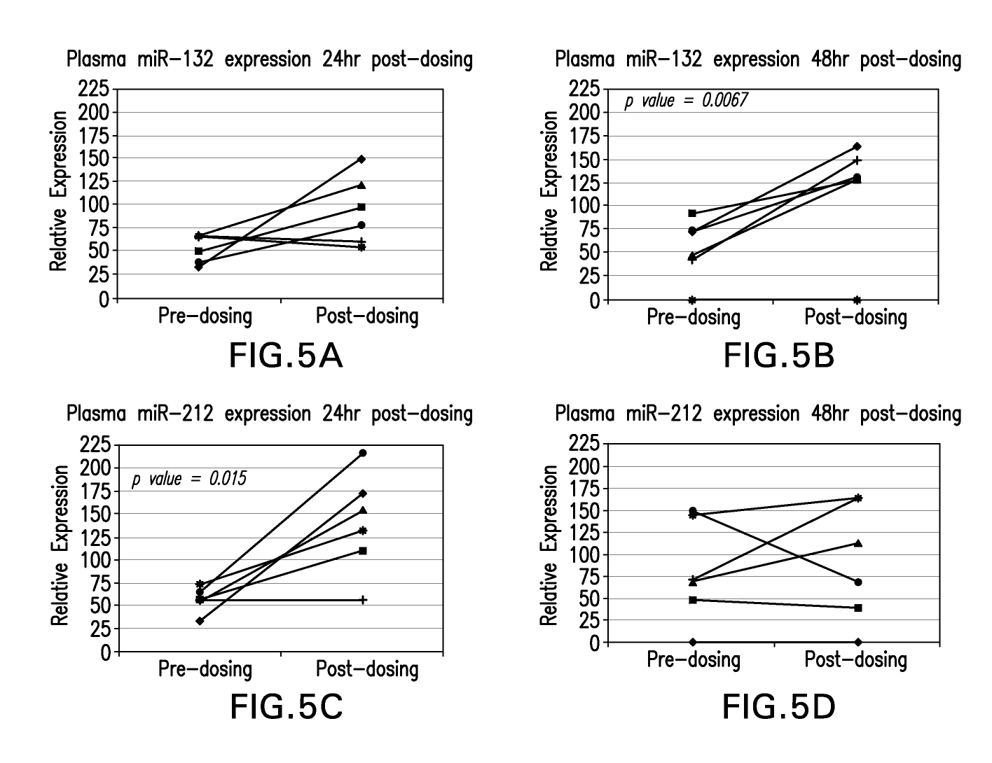

As shown in FIG. 5, plasma miR-132 levels were significantly elevated 48 hours post OXM derivative dosing (p=0.0067, n=7) and the levels for plasma miR-212 were elevated at 24 hours post-dosing (p=0.015, n=7). Conversely, the levels of miR-375 in plasma were not changed by the OXM derivative treatment.

### Example 5

The effects of other cAMP enhancing agents on miR-132 and miR-212 expression in β-cells are shown in this example.

MicroRNAs miR-132 and miR-212 are closely related miRNA species with an identical seeding region. miR-132 has been shown to be induced by cAMP in neurons (Vo et al., A cAMP-response element binding protein-induced microRNA regulates neuronal morphogenesis, Proc. Natl. Acad. Sci. USA. 102: 16426-31 (2005); Klein et al., Homeostatic regulation of MeCP2 expression by a CREB-induced microRNA, Nat. Neurosci. 10: 1513-4 (2007)). It has also been reported that there are CRE elements presented in the promoter regions of both miR-132 and miR-212 (Wu & Xie, Comparative sequence analysis reveals an intricate network among REST, CREB and miRNA in mediating neuronal gene expression, Genome Biol. 7: R85 (2006)).

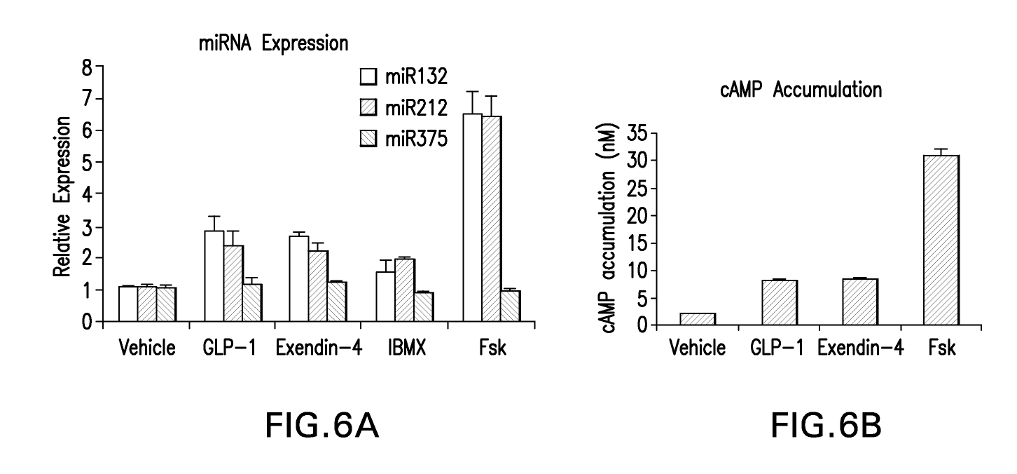

To test whether these two miRNAs were directly regulated by cAMP in β-cells, INS-1 823/3 cells were treated with several cAMP raising agents including IBMX, forskolin, and exendin-4 in addition to GLP-1. Both miR-132 and miR-212 expression was significantly increased by all treatments while miR-375 expression remained unchanged (FIG. 6A). Moreover, the fold of up-regulation was correlated with the level of cAMP accumulation by these treatments (FIG. 6B), indicating that regulation of miR-132 and miR-212 expression by GLP-1 is mediated by cAMP in β-cells. Thus, detecting and measuring these miRNAs provides a means for measuring the efficacy of any agent that raises cAMP levels in pancreatic islet β-cells.

### Example 6

Overexpression of miR-132 or miR-212 enhanced glucose-dependent insulin secretion in β-cells.

We overexpressed the precursors of miR-132 and miR-212 to evaluate their possible contribution to β-cell function. MicroRNA miR-375 was included as a control as it been reported to have an inhibitory effect on GDIS (Poy et al., Nature 432: 226-230 (2004)). Chemically modified Pre-miR™ miRNA precursor molecules and Anti-miR™ miRNA inhibitors were purchased from Ambion (Foster City, Calif.). miRCURY LNA™ microRNA knockdown oligonucleotides were purchased from Exiqon (Woburn, Mass.). The oligonucleotide molecules were delivered to the cells by the Nucleofector Device (Amaxa, Gaithersburg, Md.). In brief, INS-1 cells were trypsinized, centrifuged, and resuspended in 100 μl Nucleofector solution V. Then 100-500 pmole RNA oligonucleotides were transfected to 3-million cells at the concentration of 1-5 μM with the Amaxa Nucleofector Device. Scramble controls from the vendors were used with the same transfection method. After electroporation, the cells were transferred to regular culture medium and then split into replicates in 96-well plates. Insulin secretion assay or LANCE cAMP assay were performed 48 hours post electroporation.

For the Lance cAMP assay, INS1 832/3 and 832/13 cells were grown to near confluency in flasks. 9000 cells were incubated with GLP-1 or Exendin-4 in stimulation buffer at concentrations as indicated in figure legends for 1 hour at 37° C. cAMP levels were measured using LANCE cAMP assay kit in 384-well format according to manufacture's protocol (Perkin Elmer, Waltham, Mass.). Counts were calculated to cAMP concentrations using a cAMP standard curve. Each condition was measured in quadruplicate.

INS-1 cell glucose dependent insulin secretion (GDIS) assay was performed as follows. The GDIS assay was performed with cells grown to near confluency in 96-well plates. Prior to the assay, cells were washed once with PBS and pre-incubated for two hours in freshly prepared Krebs-Ringer Bicarbonate (KRB) medium without glucose. The medium were then replaced with KRB with 2, 8 or 16 mM glucose or along with 10 nM GLP-1. The cells were incubated for another two hours in a CO₂ incubator. The media was then removed at the end of incubation, and assayed for insulin levels by Ultra-sensitive Rat Insulin ELISA kit (ALPCO, Salem, N.H.) or insulin immunoassay by Gyrolab workstation (GYROS AB, Uppsala, Sweden). Intracellular insulin was extracted by acid ethanol method for measuring intracellular insulin content. Total protein was measured by Pierce 660 nm Protein Assay from Pierce Biotechnology, Inc. (Rockford, Ill.).

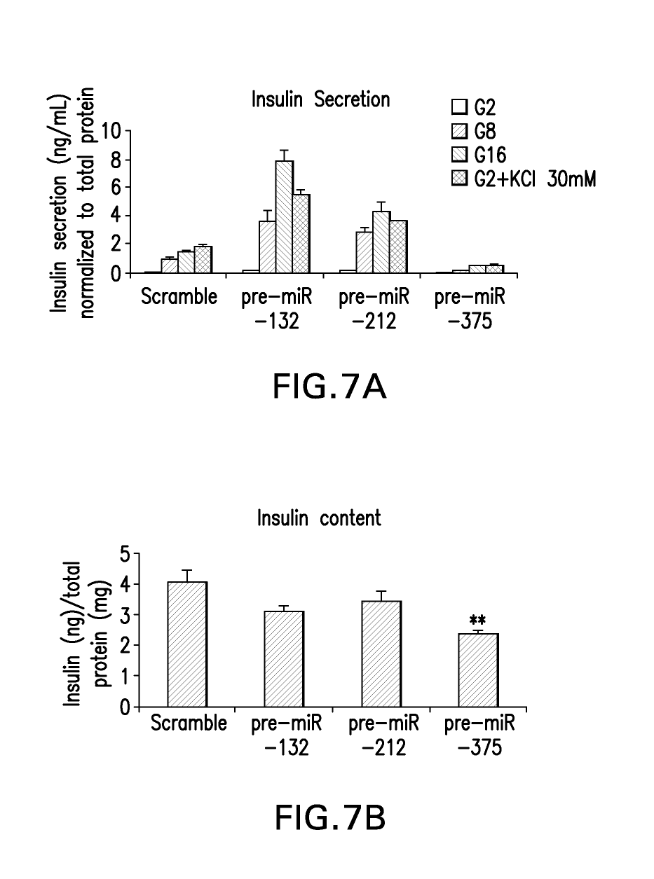

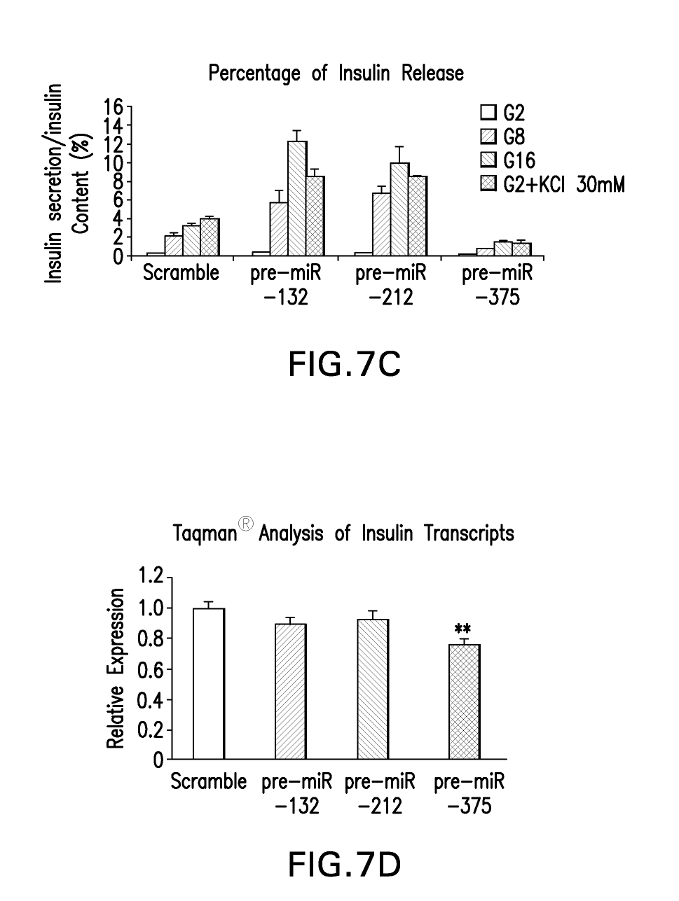

As shown in FIG. 7A, GDIS was significantly enhanced in INS-1 832/3 cells over-expressing miR-132 or miR-212, as was insulin release stimulated by KCl. However, the basal insulin secretion at low glucose was unchanged.

We further measured the insulin content and found it was unchanged by miR-132 or miR-212 overexpression (FIG. 7B). Consistent with a previous report (Poy et al., ibid.), overexpression of miR-375 in INS-1 cells significantly suppressed GDIS and resulted in a reduction of insulin content (FIGS. 7A and 7B). When insulin release was calculated as percentage of insulin secretion of intracellular insulin content, over-expression of miR-132 or miR-212 significantly increased while over-expression of miR-375 decreased insulin release (FIG. 7C). Furthermore, we measured insulin transcripts by TAQMAN analysis in cells over-expressing these microRNA molecules. Ins1 TAQMAN probe from Applied Biosystems (Foster City, Calif.) was used to measure insulin transcript levels and normalized to β-actin. Over-expressing either miR-132 or miR-212 had no effect on insulin transcripts, whereas miR-375 moderately down-regulated Ins1 transcripts (FIG. 7D). In this series of experiments, miR-132, miR-212 and miR-375 transcripts were increased by about 1000-fold, 2000-fold, and 8-fold, respectively, as assayed by TAQMAN real-time quantitative RT-PCR.

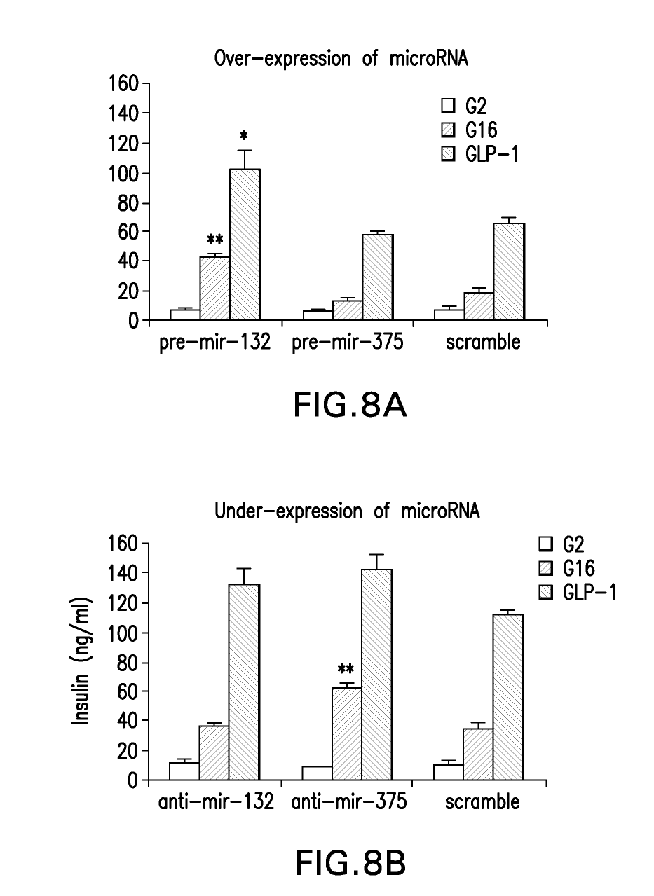

Next we examined the effect of miR-132 and miR-212 on GLP-1 potentiation of GDIS. GLP-1 potentiation was robustly increased by overexpressing miR-132 but not miR-375 compared to the scramble control (FIG. 8A). Overexpression of miR-212 also resulted in enhanced GLP-1 potentiation (data not shown). In parallel, we investigated the loss of function of these microRNAs on GDIS and GLP-1 potentiation by using sequence-specific antisense oligonucleotides.

Due to the very low endogenous expression levels of miR-212 in INS-1 832/3 cells, we were not able to reliably quantify the knockdown efficiency of this miRNA. Therefore, we focused on studying the effect of knockdown of miR-132. First, we observed no change in GDIS or GLP-1 potentiation when introducing anti-miR-132 inhibitors, whereas anti-miR-375 inhibitors significantly increased GDIS (FIG. 8B). In this loss-of-function experiment, the knock down efficiency for miR-132 and miR-375 was 42% and 45% respectively. Furthermore, we observed no measurable effect on GDIS by knocking down 80% of endogenous miR-132 by transfecting five fold excess anti-miR-132 inhibitors.

Next, we used another antisense reagent, miRCURY LNA miR-132 knockdown molecules, to repeat the experiment at two doses. No effect was found on GDIS or the insulinotropic effect of GLP-1 with 50-60% knock down efficiency.

Taken together, the results of the gain-of-function and loss-of-function experiments suggested that miR-132 and miR-212 are sufficient but not necessary for enhancing GDIS and GLP-1 potentiation in β-cells.

### Example 7

As shown in this example, GLP-1 does not regulate miR-132 and miR-212 expression in INS-1 832/13 cells.

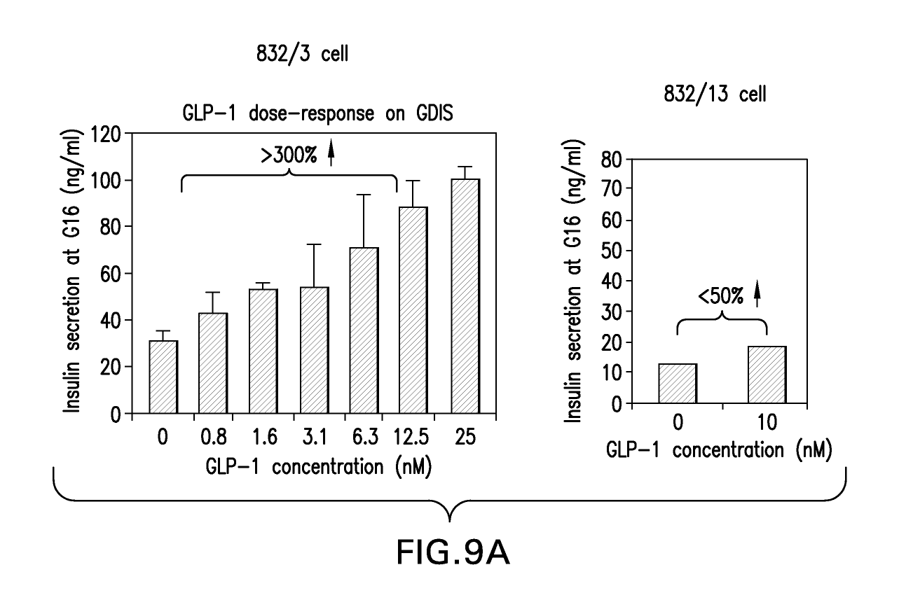

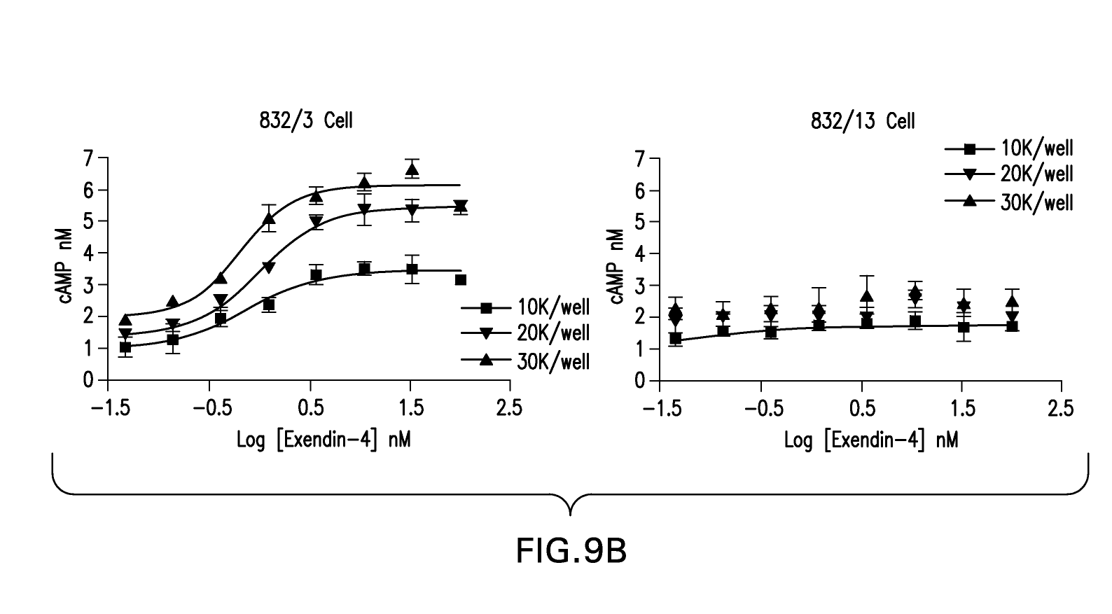

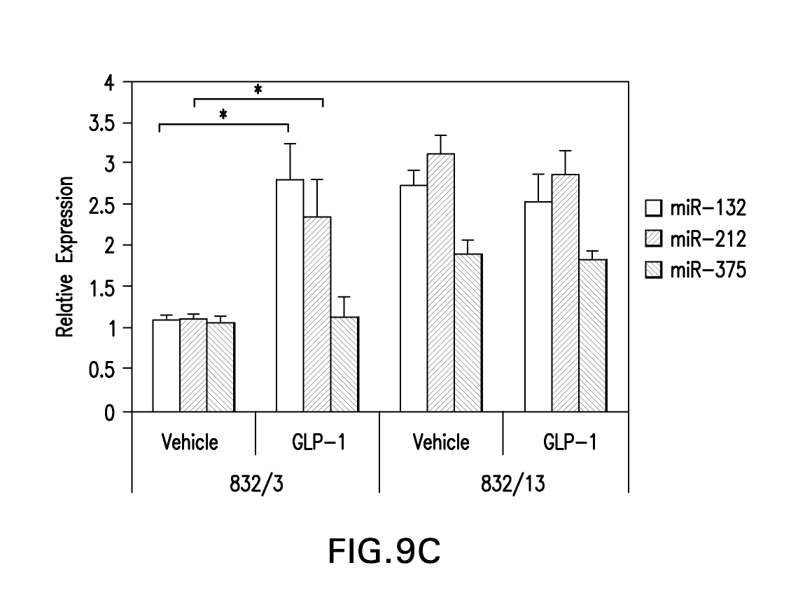

INS-1 832/3 and INS-1 832/13 cells are two lines with distinct responsiveness to GLP-1. As previously reported (Ronnebaum et al., J. Biol. Chem. 283: 28909-17 (2008)), we observed greater than 300% increase of insulin secretion upon treatment with GLP-1 in the INS-1 832/3 line but less than 50% increase in INS-1 832/13 cells (FIG. 9A).

Since GLP-1R couples to Gas and GLP-1 signaling induces cAMP accumulation, we hypothesized that the cAMP regulation in 832/13 cells would be less responsive to GLP-1 than in the INS-1 832/3 cells. To test this, both cell lines were stimulated with the GLP-1 analog Exendin-4 for one hour. We observed dose-dependent accumulation of cAMP in the INS-1 832/3 cells and a loss of such response in INS-1 832/13 cells (FIG. 9B).

Next we examined the responsiveness of miR-132 and miR-212 expression by GLP-1 in INS-1 832/13 cells by TAQMAN analysis. Unlike in INS-1 832/3 cells where GLP-1 treatment significantly increased the expression of miR-132 and miR-212, such a response to GLP-1 was not observed in 832/13 cells (FIG. 9C). The induction of miR-132/212 by GLP-1 is largely omitted in the INS-1 832/13 cells, which directly correlates with the lack of cAMP accumulation and insulin secretory responses to GLP-1 in this sibling line of the INS-1 cells.

### Example 8

Overexpression of miR-132 and miR-212 partially restored GLP-1 potentiation in INS-1 832/13 cells.

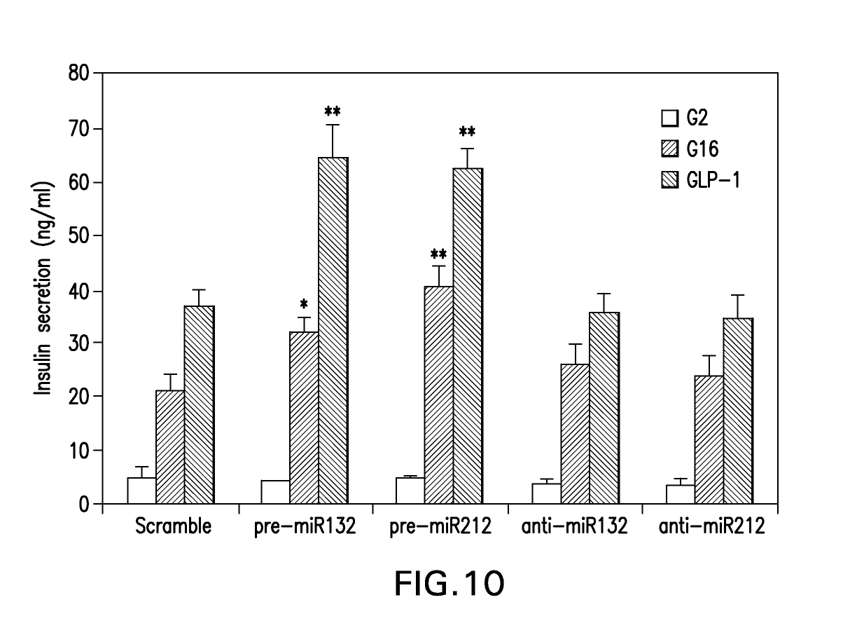

The correlation of the lack of induction of miR-132/212 by GLP-1 in the INS-1 832/13 cells with the lack of cAMP accumulation and insulin secretory responses to GLP-1 suggested that miR-132/212 may be mediators for GLP-1's insulinotropic effect. We therefore tested whether overexpression of miR-132 or miR-212 in INS-1 832/13 cells could restore GLP-1 potentiation in INS-1 832/13 cells. When pre-miR-132 or pre-miR-212 precursors were overexpressed, GDIS was significantly increased as observed in INS-1 832/3 cells (FIG. 10). Furthermore, the effect of GLP-1 was also augmented compared to the scramble control (FIG. 10).

We measured cAMP accumulation in INS-1 832/13 cells overexpressing pre-miR-132 or pre-miR-212 and found no changes in cAMP levels compared to the scramble control suggesting that these two miRNAs themselves do not regulate cAMP levels in β-cells. Lastly, we introduced anti-sense inhibitors of miR-132 and miR-212 into INS-1 832/13 cells and observed no changes in insulin secretion: consistent with what was found in the INS-1 832/3 cells (FIG. 10). The data showing the loss of regulation of miR-132 and 212 by GLP-1 and the rescue of GLP-1 potentiation in INS-1 832/13 cells by over-expressing miR-132 and miR-212 together suggested that GLP-1's insulinotropic effect was mediated in part by miR-132 and 212 in β-cells.

### Example 9

This example provides a prophetic example of an assay that can be used to detect and quantitate circulating levels of miR-132 and/or miR-212 in a blood sample obtained from a subject being administered a GLP-1 receptor agonist or other agent that increases cAMP levels in pancreatic islet cells.

Examples of probes and primers that can be used are shown in Table 1. The miR-132 and miR-212 probes and primers have also been disclosed in Yuen et al., Molec. Cell. Endocrinol. 302: 12-17 (2009). The RT linker probe forms a loop stem structure and has 3′ end nucleotide sequence that is complementary to the last 6-8 nucleotides of the miRNA. The detector probes have a 5′ sequence that corresponds in sequence to the 3′ end of the RT linker probe in the stem region and about 6-8 nucleotides at the 3′ end that are complementary to the terminal 6-8 nucleotides of the miRNA. In this example, the detector probe has a 6-FAM (6-carboxyfluorescein) fluorophore at the 5′ end and an MGB (minor groove binder) ligand conjugated to TAMRA (tetramethylrhodamine) quencher at the 3′ end. The forward primers have a nucleotide sequence that is corresponds to the 5′ end of the miRNA and is complementary to the last several nucleotides at the 3′ end of the detector probe and short 5′ nucleotide extensions. The reverse primer is a universal primer and has a nucleotide sequence that corresponds to a nucleotide sequence that spans the stem-loop region of the RT linker probe. Thus, it can be used in assays to detect miR-132 or miR-212 or any other miRNA that includes same nucleotide sequence.

TABLE 1

| | | SEQ ID |
|---|---|---|
| Description | Sequence | NO: |
| miR-132/RT linker probe | 5′-GTCGTATCCAGTGCAGGGTCCGAGGTATT | 17 |
| | CGCACTGGATACGACCGACCA-3′ | |
| miR-132/Forward primer | 5′-GCCGCTAACAGTCTACAGCCAT-3′ | 18 |
| miR-132/Reverse primer | 5′-GTGCAGGGTCCGAGGT-3′ | 19 |
| miR-132/Detector probe | 5′-FAM-TGGATACGACCGACCAT-MGB-3 | 20 |
| miR-212/RT linker probe | 5′-GTCGTATCCAGTGCAGGGTCCGAGGTAG | 21 |
| | GCGCACTGGATACGACGGCCGT-3′ | |
| miR-212/Forward primer | 5′-GCCGCTAACAGTCTCCAGTCA-3′ | 22 |
| miR-212/Detector probe | 5′-FAM-TGGATACGAC GGCCGTG-MGB-3′ | 23 |
| miR-375/RT linker probe | 5′-GTCGTATCCAGTGCAGGGTCCGAGGTAG | 24 |
| | GCGCACTGGATACGACGGCCGTTCACG-3′ | |
| miR-375/Forward primer | 5′-GGCCTTTGTTCGTTCGGCT-3′ | 25 |
| miR-375/ Detector probe | 5′-TGGATACGACTCACGCA-3′ | 26 |

Approximately 50 μL blood each is collected into EDTA tubes before and then at time points thereafter (for example, 24 hours and 48 hours post-administration). The blood samples are immediately placed on ice and plasma prepared within one hour and stored at −80° C. In general, about 50 μL of the plasma sample is thawed on ice and 2× Denaturing Solution (Ambion) is added. To allow for normalization for sample variations, a miRNA such as the C. elegans miRNAs cel-miR-39, cel-miR-54, or cel-miR-238, can be added at a final concentration of 20, 200, and 2000 fM (sequence information according to miRBase). MicroRNA miR132, miR-212, and miR-375 expression levels are normalized to spiked-in C. elegans miRNA. Primers and probes to any of the above miRNAs can be obtained from Applied Biosystems. RNA extractions can be performed as described in Example 3.

Relative miRNA levels are determined by real-time reverse transcription PCR reaction using the ABI PRISM 7900 Sequence Detection System from Applied Biosystems (Foster City, Calif.) according to the manufacturer's protocol. Briefly, 6 μL total RNA is mixed with 1 U MultiScribe Reverse Transcriptase (Applied Biosystems), 0.25 U RNase Inhibitor, 2 μL hairpin-looped miRNA-specific RT linker probe, 1 mM dNTPs and 1× Reverse Transcription Buffer (Applied Biosystems) in a total volume of 10 μL. The mixture is incubated at 16° C. for 30 minutes, 42° C. for another 30 minutes, and the reaction is stopped by heating to 85° C. for five minutes.

Real time PCR reaction can be set up in a 20 μL volume containing 1.33 μL first strand cDNA, 1× TAQMAN MicroRNA Assay Mix (Applied Biosystems) containing forward and reverse primers and detector probe and 1× TAQMAN Universal PCR Master Mix (Applied Biosystems). After activation of the AMPLITAQ Gold DNA polymerase at 95° C. for 10 minutes, 40 cycles of two-step PCR were run (95° C. for 15 seconds and 60° C. for 60 seconds). Data is collected and analyzed with SDS v.2.2.2 software (Applied Biosystems) or equivalent.

Detection of an increase in the level of miR-132 and/or miR-212 in the blood samples indicates that the agent has engaged the pancreatic islet cells. The circulating levels of the miRNAs can be used to correlate drug levels and changes in glucose and insulin/C-peptide levels, which in turn reflect the engagement of the pancreatic islet cells by the agent.

SEQUENCES

| SEQ ID | | |
|---|---|---|
| NO: | Description | Sequence |
| 1 | Rattus norvegicus (Rat) | CCGCCCCCGCGUCUCCAGGGCAACCGUGGCUU |
| | miR-132 stem loop: rno- | UCGAUUGUUACUGUGGGAACCGGAGGUAACA |
| | mir-132 MI0000905 (nts | GUCUACAGCCAUGGUCGCCCCGCAGCACGCCC |
| | 59-80 mature sequence) | ACGCU C |
| 2 | Rattus norvegicus (Rat) | CGGGAUAUCCCCGCCCGGGCAGCGCGCCGG |
| | miR-212 stem loop: rno- | CACCUUGGCUCUAGACUGCUUACUGCCCGG |
| | mir-212 MI0000952 (nts | GCCGCCCUCAGUAACAGUCUCCAGUCACGG |
| | 72-93 mature sequence) | CCACCGACGCCUGGCCCCGCC |
| 3 | Homo sapiens (human) | CCGCCCCCGCGUCUCCAGGGCAACCGUGGCUU |
| | miR-132 stem loop: hsa- | UCGAUUGUUACUGUGGGAACUGGAGGUAACA |
| | mir-132 MI0000449 (nts | GUCUACAGCCAUGGUCGCCCCGCAGCACGCCC |
| | 59-80 mature sequence) | ACGCGC |
| 4 | Homo sapiens (human) | CGGGGCACCCCGCCCGGACAGCGCGCCGGCAC |
| | miR-212 stem loop: hsa- | CUUGGCUCUAGACUGCUUACUGCCCGGGCCGC |
| | mir-212 MI0000288 (nts | CCUCAGUAACAGUCUCCAGUCACGGCCACCGA |
| | 71-91 mature sequence) | CGCCUGGCCCCGCC |
| 5 | Mus musculus (mouse) | GGGCAACCGUGGCUUUCGAUUGUUACUGUGGG |
| | miR-132 stem loop: mmu- | AACCGGAGGUAACAGUCUACAGCCAUGGUCGC |
| | mir-132 MI0000158 (nts | CC |
| | 42-63 mature sequence) | |
| 6 | Mus musculus (mouse) | GGGCAGCGCGCCGGCACCUUGGCUCUAGACUG |
| | miR-212 stem loop: mmu- | CUUACUGCCCGGGCCGCCUUCAGUAACAGUCU |
| | mir-212 MI0000696 (nts | CCAGUCACGGCCACCGACGCCUGGCCC |
| | 56-77 mature sequence) | |
| 7 | Macaca mulatta (rhesus | CCGCCCCCGCGUCUCCAGGGCAACCGUGGCUU |
| | monkey) miR-132 stem | UCGAUUGUUACUGUGGGAACUGGAGGUAACA |
| | loop: mml-mir-132 | GUCUACAGCCAUGGUCGCCCCGCAGCACGCCC |
| | MI0007620 (nts 59-80 | ACGCGC |
| | mature sequence) | |
| 8 | Macaca mulatta (rhesus | CGGGGCACCCCGCCCGGACAGCGCGCCGGCAC |
| | monkey) miR-212 stem | CUUGGCUCUAGACUGCUUACUGCCCGGGCCGC |
| | loop: mml-mir-212 | CCUCAGUAACAGUCUCCAGUCAGGGCCACCGA |
| | MI0007671 (nts 71-91 | CGCCUGGCCCCGCC |
| | mature sequence) | |
| 9 | Canis familiaris (dog) | AACCGUGGCUUUCGAUUGUUACUGUGGGAACC |
| | miR-132: cfa-mir-132 | GGAGGUAACAGUCUACAGCCAUGGUCGC |
| | MI0008156 (nts 38-60 | |
| | mature sequence) | |
| 10 | Canis familiaris (dog) | ACCUUGGCUCUAGACUGCUUACUGCCCGGGC |
| | miR-212 stem loop: | CGCCCUCAGUAACAGUCUCCAGUCACGGCCA |
| | MI0008155 (nts 1-23 | |
| | mature sequence) | |
| 11 | Monodelphis domestica | GGGCAACCGUGGCUUUCGAUUGUUACUGUGGG |
| | (short-tailed opossum) | AACCAGGGGUAACAGUCUACAGCCAUGGUCGC |
| | miR-132 stem loop: mdo- | CC |
| | mir-132 MI0005338 (nts | |
| | 42-63 mature sequence) | |
| 12 | Monodelphis domestica | GGGCAGCGCGCCGGCACCUUGGCUCUAGACUG |
| | (short-tailed opossum) | CUUACUGCCCGGGCCACCCUCAGUAACAGUCU |
| | miR-212 stem loop: mdo- | CCAGUCACGGCCACCGACGCCUGGCCC |
| | mir-212 MI0005337 (nts | |
| | 56-76 mature sequence) | |
| 13 | Mature miR-132 | UAACAGUCUACAGCCAUGGUCG |
| 14 | Mature miR-212 | UAACAGUCUCCAGUCACGGCC |
| 15 | Mature miR-217 | UACUGCAUCAGGAACUGAUUGGAU |
| 16 | Mature miR-375 | UUUGUUCGUUCGGCUCGCGUGA |
| 17 | miR-132/RT linker probe | GTCGTATCCAGTGCAGGGTCCGAGGTATTCGCA |
| | | CTGGATACGACCGACCA |
| 18 | miR-132/Forward primer | GCCGCTAACAGTCTACAGCCAT |
| 19 | miR-132/Reverse primer | GTGCAGGGTCCGAGGT |
| 20 | miR-132/detector probe | TGGATACGACCGACCAT |
| 21 | miR-212/RT linker probe | GTCGTATCCAGTGCAGGGTCCGAGGTAGGCGCA |
| | | CTGGATACGACGGCCGT |
| 22 | miR-212/Forward primer | GCCGCTAACAGTCTCCAGTCA |
| 23 | miR-212/detector probe | TGGATACGACGGCCGTG |
| 24 | miR-375/RT linker probe | 5′-GTCGTATCCAGTGCAGGGTCCGAGGTAGGCG |
| | | CACTGGATACGACGGCCGTTCACG |
| 25 | miR-375/Forward primer | GGCCTTTGTTCGTTCGGCT |
| 26 | miR-375/ Detector probe | TGGATACGACTCACGCA |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for determining whether a treatment for a metabolic disorder that includes an agent that effects an increase in intracellular cAMP in pancreatic islet β-cells is engaging the pancreatic islet β-cells in a subject, comprising:
measuring the level of at least one of an miRNA selected from the group consisting of miR-132 or miR-212 in a test sample from the subject, wherein an increase in the level of the miRNA in the test sample relative to the level of the corresponding miRNA in a control sample indicates the treatment is engaging the pancreatic islet β-cells.

2. The method of claim 1, wherein the miRNA is detected using reverse-transcription polymerase chain reaction (RT-PCR).

3. The method of claim 2, wherein the RT-PCR comprises obtaining total RNA from the test sample, adding the total RNA to a reaction mixture comprising
(a) a linker probe having a stem, a loop, and a 3′ end sequence that base pairs with a 3′ end sequence of the miRNA, allowing the linker probe to hybridize the miR-132, and extending the linker probe to form an extension reaction product;
(b) amplifying the extension product to produce an amplification product in a polymerase chain reaction comprising a forward primer that hybridizes to the 5′ region of the miR-RNA sequence in the extension or amplification product or a complementary sequence to the 5′ region of the miR-RNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and
(c) detecting the detectable signal wherein the presence of the detectable signal relative to the level of the detectable signal in the control reaction indicates the agent is engaging the pancreatic islet β-cells.

4. The method of claim 1, wherein the test sample is whole blood, plasma, or serum.

5. The method of claim 1, wherein the agent is selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, GPR-119 receptor agonist, phosphodiesterase inhibitor, dipeptidyl peptidase (DPP IV) inhibitor, and combinations thereof.

6. The method of claim 1, wherein the metabolic disorder is metabolic syndrome, obesity, diabetes (type I or type II), metabolic syndrome X, hyperglycemia, impaired fasting glucose, dyslipidemia, atherosclerosis, or other prediabetic state.

7. A method for determining the efficacy of a treatment regimen for diabetes comprising:
(a) obtaining a body fluid sample from a subject undergoing the treatment regimen; and
(b) measuring the level of at least one of an miRNA selected from the group consisting of miR-132 or miR-212 in a test sample from the subject, wherein an increase in the level of the miRNA in the test sample relative to the level of the corresponding miRNA in a control sample indicates the treatment is efficacious.

8. The method of claim 7, wherein the miRNA is detected using reverse-transcription polymerase chain reaction (RT-PCR).

9. The method of claim 8, wherein the RT-PCR comprises obtaining total RNA from the test sample, adding the total RNA to a reaction mixture comprising
(a) a linker probe having a stem, a loop, and a 3′ end sequence that base pairs with a 3′ end sequence of the miRNA, allowing the linker probe to hybridize the miR-132, and extending the linker probe to form an extension reaction product;
(b) amplifying the extension product to produce an amplification product in a polymerase chain reaction comprising a forward primer that hybridizes to the 5′ region of the miR-RNA sequence in the extension or amplification product or a complementary sequence to the 5′ region of the miR-RNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and
(c) detecting the detectable signal wherein the presence of the detectable signal relative to the level of the detectable signal in the control reaction indicates the treatment is efficacious.

10. The method of claim 7, wherein the test sample is whole blood, plasma, or serum.

11. The method of claim 7, wherein the treatment comprises administering to the subject a glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, GPR-119 receptor agonist, phosphodiesterase inhibitor, dipeptidyl peptidase (DPP IV) inhibitor, and combinations thereof.

12. The method of claim 7, wherein the metabolic disorder is metabolic syndrome, obesity, diabetes (type I or type II), metabolic syndrome X, hyperglycemia, impaired fasting glucose, dyslipidemia, atherosclerosis, or other prediabetic state.

13-24. (canceled)

25. A method for identifying an agent for treating a metabolic disorder that targets a receptor in pancreatic islet β-cells that raises intracellular levels of cAMP, comprising:
measuring the level of at least one of an miRNA selected from the group consisting of miR-132 or miR-212 in a test sample obtained from a subject administered the agent, wherein an increase in the level of the miRNA in the test sample relative to the level of the corresponding miRNA in a control sample indicates the agent is targeting the receptor in the pancreatic islet β-cells that raises cAMP levels in the pancreatic islet β-cells.

26. The method of claim 25, wherein the miRNA is detected using reverse-transcription polymerase chain reaction (RT-PCR).

27. The method of claim 26, wherein the RT-PCR comprises obtaining total RNA from the test sample, adding the total RNA to a reaction mixture comprising
(a) a linker probe having a stem, a loop, and a 3′ end sequence that base pairs with a 3′ end sequence of the miRNA, allowing the linker probe to hybridize the miR-132, and extending the linker probe to form an extension reaction product;
(b) amplifying the extension product to produce an amplification product in a polymerase chain reaction comprising a forward primer that hybridizes to the 5′ region of the miR-RNA sequence in the extension or amplification product or a complementary sequence to the 5′ region of the miR-RNA sequence in the extension or amplification product, a reverse primer that hybridizes to the linker probe sequence in the extension or amplification product or a complementary sequence to the linker probe sequence in the extension or amplification product, and a detector probe that hybridizes to a nucleotide sequence of the linker probe stem sequence in the extension or amplification product or hybridizes to a nucleotide sequence of a complementary sequence of the linker probe stem sequence in the extension or amplification product and which produces a detectable signal; and
(c) detecting the detectable signal wherein the presence of the detectable signal relative to the level of the detectable signal in the control reaction indicates the agent is targeting the receptor in the pancreatic islet β-cells that raises cAMP levels in the pancreatic islet β-cells.

28. The method of claim 25, wherein the test sample is whole blood, plasma, or serum.

29. The method of claim 25, wherein the agent is selected from the group consisting of glucagon-like peptide-1 (GLP-1), glucagon-like peptide analog (GLP-1 analog), glucagon-like peptide derivative (GLP-1 derivative), glucose-dependent insulinotropic polypeptide (GIP), glucose-dependent insulinotropic polypeptide (GIP) derivative, glucose-dependent insulinotropic polypeptide (GIP) analog, oxyntomodulin, oxyntomodulin derivative, oxyntomodulin analog, exendin peptide, exendin peptide derivative, exendin peptide analog, glucagon peptide, glucagon peptide derivative, glucagon peptide analog, GRP-119 receptor agonist, phosphodiesterase inhibitor, dipeptidyl peptidase (DPP IV) inhibitor, and combinations thereof.

30. The method of claim 25, wherein the metabolic disorder is metabolic syndrome, obesity, diabetes (type I or type II), metabolic syndrome X, hyperglycemia, impaired fasting glucose, dyslipidemia, atherosclerosis, or other prediabetic state.
